(19) United States
(12) Patent Application Publication
(10) Pub. No.: US 2014/0199243 A1
(43) Pub. Date: Jul. 17, 2014

(54) BIFUNCTIONAL PHOSPHONATE CHELATING AGENTS

(71) Applicant: CENTRE NATIONAL DE LA RECHERCHE SCIENTIFIQUE, Paris Cedex 16 (FR)

(72) Inventors: Loic Charbonniere, Weyersheim (FR); Câline Christine, Schiltigheim (FR); Alexandre Lecointre, Fessenheim-le-bas (FR); Katia Nchmimi Nono, Strasbourg (FR)

(73) Assignee: CENTRE NATIONAL DE LA RECHERCHE SCIENTIFIQUE, Paris Cedex 16 (FR)

(21) Appl. No.: 14/126,486

(22) PCT Filed: Jun. 18, 2012
(86) PCT No.: PCT/FR2012/051360

§ 371 Date: Feb. 12, 2014

(30) Foreign Application Priority Data
Jun. 17, 2011 (FR) 11/55337
Oct. 12, 2011 (FR) 11/59210

(51) Int. Cl. A61K 49/00

(52) U.S. Cl. CPC A61K 49/0002
USPC 424/9.1; 546/24; 530/391.5; 530/409

(57) ABSTRACT

Compounds of the following Formula (I):

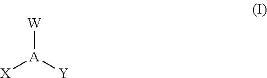

in which
A represents either a nitrogen atom, or a ring having 3 to 6 carbon atoms, or an aromatic ring having 5 to 10 members, the ring and the aromatic ring optionally include one or more heteroatoms selected from N, O and S, and W represents a grafting function and X and Y represent chelating functions.

The present invention relates to bifunctional compounds, the complexes formed from said compounds and the therapeutic or diagnostic use of said complexes.

The application of metallopharmaceutical diagnostic and therapeutic agents is becoming more and more important in biological and medical research, as well as in diagnostic and therapeutic procedures. In general, these agents contain a radioisotope or paramagnetic or luminescent metal, which, when introduced into a subject, becomes localized on a previously selected target: organ, tissue or part of the skeleton. In the case of a diagnostic method, images representing the distribution in vivo of the radioisotope, paramagnetic or radiopaque or luminescent metal can be formed by various means, including single photon emission, magnetic resonance and X-rays, depending on the metal selected and the model of substitution on the metallic complex. The distribution and corresponding relative intensity of the radioisotopes or of the paramagnetic or radiopaque or luminescent metal detected indicate not only the space occupied by the target tissue, but can also indicate the presence of receptors, antigens, aberrations, pathological conditions, etc. In the context of a therapeutic method, the agent generally contains a radioisotope and delivers a dose of radiation locally to the selected site.

Depending on the target organ or tissue of interest and depending on whether it is in the context of a diagnosis or a therapeutic treatment, a range of metallopharmaceutical agents can be used. In general these complexes are in the form of a conjugate comprising a radioactive or paramagnetic or luminescent metal, a supporting agent for targeting conjugated to an organ or a specific site of the tissue, and a bond for chemically binding the metal to the transporter.

Positron emission tomography (PET) is a non-invasive medical imaging technique used for diagnosing certain cancers, and for monitoring the development of tumours and the efficacy of treatments. The images are obtained by injecting a positron-emitting radioactive tracer (radioactive molecule) into the organism.

Copper ⁶⁴Cu is increasingly of interest in the field of medical imaging. In fact, the lifetime of this positron-emitting radioelement is significantly greater than that of fluorine ¹⁸F, the radioisotope most commonly used. Copper ⁶⁷Cu, owing to its gamma-emitting property, is of interest in therapeutic nuclear medicine by internal radiotherapy (local irradiation of the tumour in a restricted perimeter).

In order to be used, the ⁶⁴Cu and ⁶⁷Cu must be incorporated in a bifunctional chelating agent (BCA). This double functionality makes it possible on the one hand to bind the metal, and on the other hand to create a covalent bond with a biologically active macromolecule or vector, capable for example of targeting the receptors overexpressed in certain diseases, in particular cancers.

The complexing agents that are the most developed are acyclic chelating agents of the polyaminocarboxylate type or macrocyclic chelating agents of the tetraaza-, polyaminocarboxylate and polyaminophosphonate type. However, these copper chelating agents have only moderate stability in vivo, low stability with respect to an acid environment and undergo a phenomenon of reduction which leads to loss of the metal.

Thus, Smith et al. (Journal of Inorganic Biochemistry, (2004), 98, 1874-1901) review BCAs and their use for radiolabelling with ⁶⁴Cu and ⁶⁷Cu. The BCAs mentioned are either polyaminocarboxylic or polyaminophosphonate macrocycles, or open polyaminocarboxylates. The latter have far lower thermodynamic stability than the macrocycles.

Recently, patent application US 2010019627 described radiopharmaceutical agents comprising a copper-chelating macrocyclic bifunctional agent based on sarcophagine bearing carboxylic acid functions.

Mezzaros et al. (Inorganica Chimica Acta, (2010), 363, 1059-1069) describe BCAs based on hydrazinonicotinamide (HYNIC), a chelator of Tc-99 m. HYNIC, used in the form of an activated ester, in particular in the form of an N-hydrosuccinimide derivative, binds to the biomolecules by an amide bond formed between the activated ester function and the amine functions of said biomolecule. However, for coordination with the metal it is necessary to use an additional co-ligand, which makes the final structure of the complex, and therefore its use, uncertain.

Thus, there is a need for bifunctional ligands that make it possible to obtain stable complexes and avoid the release of the metals in the organism.

Application EP 0298939 describes bifunctional ligands derived from pyridines that are said to be more stable than the ligands used conventionally, but the chelating functions of these new ligands are still carboxylate groups.

Now, the inventors have shown that monofunctional ligands based either on a 2,6-bis[(N,N-bis(methylene phosphonic acid)aminomethyl]pyridine unit, or on a bispyrazolylpyridyl unit, form complexes with the metal cations that are very stable, and in particular much more stable than their carboxylated homologues (Abada S. et al., Dalton Trans (2010), 39, 9055-9062 and Nchnimi Nono et al. Inorganic Chemistry, 2011, 50, No. 5, 1689-1697).

The purpose of the present invention is therefore to propose bifunctional complexes that have improved properties relative to the bifunctional complexes of the prior art, in particular better stability. Accordingly, a subject of the invention is novel compounds, use thereof for preparing complexes with metal ions, for preparing conjugates with biological macromolecules or carriers as well as the use of the complexes and conjugates obtained as markers, as relaxation agents for NMR, for MRI imaging, for positron emission tomography (PET) and single-photon emission tomography (SPET), for luminescence microscopy, for fluoro-immunological analyses and as medicaments.

A subject of the present invention is therefore compounds of the following Formula (I):

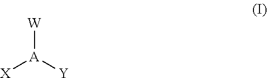

in which
A represents
   either a nitrogen atom,
   or a ring comprising from 3 to 6 carbon atoms, or an aromatic ring comprising from 5 to 10 members, said ring and said aromatic ring optionally comprising one or more heteroatoms selected from N, O and S,
W represents
   either a bromine atom, or an iodine atom,
   or an E-G-Q group with
      E selected from the group comprising an oxygen atom, a —C≡C— group, a (CH₂)ₘ group, m being an integer comprised between 0 and 5, and a —CONH— group,
      G is selected from the group comprising
         i) —(CH₂)ₒ, o being an integer comprised between 0 and 5,
         ii) —(CH₂)ₙ—NH—, n being an integer comprised between 0 and 5,
         iii) —(CH₂)p-CO—NH—(CH₂)q-NH—, p and q being integers comprised between 0 and 5, and
         iv)

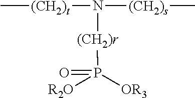

r, s and t being, each independently of one another, an integer comprised between 0 and 5, and R₂ and R₃ representing, each independently of one another, a hydrogen atom, a (C₁-C₄)alkyl group or a hydrolyzable group,
Q represents either a hydrogen atom, or an amine protecting group, or a functional group capable of forming a covalent bond with the primary and secondary amines, alcohols and thiols and
X and Y represent, independently of one another,
   either a hydrogen atom,
   or a

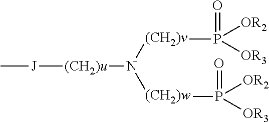

group
where u is an integer equal to 0 or 1, v and w, identical or different, are integers equal to 1 or 2, R₂ and R₃ represent, each independently of one another, a hydrogen atom, a (C₁-C₄)alkyl group or a hydrolyzable group selected from the group comprising the esters and the amides and J is either —CH₂, or is an aromatic ring comprising 5 to 10 members and optionally one or more heteroatoms selected from N, O and S,
   or a

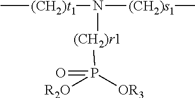

group, where r₁, s₁ and t₁ are, each independently of one another, an integer comprised between 1 and 2, and R₂ and R₃ represent, each independently of one another, a hydrogen atom, a (C₁-C₄)alkyl group or a hydrolyzable group,
provided that X, W and Y are not simultaneously H,
and salts thereof.

These compounds of Formula (I) are bifunctional ligands comprising both phosphonated chelating functions and a grafting function. The terms compounds of Formula (I) and bifunctional ligands are used indiscriminately.

Within the meaning of the present invention, by ring comprising from 3 to 6 carbon atoms is meant any saturated carbon ring comprising a skeleton of 3, or 4 or 5, or 6 carbon atoms. In said ring, one or more carbon atoms can be substituted by other atoms different from carbon, selected from nitrogen, oxygen or sulphur atoms. By way of example, the cyclopropyl, cyclobutyl, cyclopentyl, cyclohexyl, oxirane, aziridine, tetrahydrothiophene, tetrahydrofuran, piperidine, dioxane, pyrrolidine, morpholine and oxathiolane groups may be mentioned.

By aromatic ring is meant any unsaturated carbon ring comprising a skeleton of 5, or 6, or 7 or 8 or 9 or 10 carbon atoms. In said ring, one or more carbon atoms can be substituted by other atoms different from carbon, selected from nitrogen, oxygen or sulphur atoms. By way of example, phenyl, benzyl, naphthyl, pyran, pyrrole, thiophene, furan, pyridine, pyrimidine, pyrazine, triazine, imidazole, thiazole, oxazole, purine, pyrazole, triazole, tetrazole, thiadiazole, and benzothiazole may be mentioned.

By (C₁-C₄)alkyl group is meant a linear or branched group comprising from 1 to 4 carbon atoms selected from the group comprising the methyl, propyl, n-butyl, isobutyl or tert-butyl groups.

By hydrolyzable group is meant a group that can be hydrolysed in situ to give a free OH function; as examples: (a) the —COOR ester groups in which R represents either a (C₁-C₄)alkyl group as defined above, or an aryl group, in particular a phenyl or benzyl group, (b) the —CONRR′ amide groups in which R and R′ represent, each independently of one another, either a hydrogen atom, or a (C₁-C₄)alkyl group as defined above, or an aryl group, in particular a phenyl or benzyl group, (c) the —R—O—R′ ether groups in which R and R′ represent, each independently of one another, either a (C₁-C₄)alkyl group as defined above, or an aryl group, in particular a phenyl or benzyl group may be mentioned.

By amine protecting group is meant the groups used conventionally, in particular the Boc, Fmoc, acyl, trifluoroacetamide, benzyl, tosyl and phthalimide groups.

The functional group capable of forming a covalent bond with the primary and secondary amines, alcohols and thiols of a biologically active molecule or of a carrier will be selected as a function of these various groups and said selection is within the capabilities of a person skilled in the art. Thus, if this functional group and the group with which it is to react are both electrophilic groups or both nucleophilic groups, then an oxidative coupling can be carried out in order to form the bond (for example —SH+HS→—SS—) or one of the two groups can be converted chemically to a group of the opposite type by activating for example the bifunctional coupling reagents. If this functional group is a nucleophilic group and the group with which it is to react is an electrophilic group or vice versa, these two groups can generally be reacted with one another without any prior activation. Thus, if the active molecule is a protein with free amine groups, then this functional group will be able to bear an activated acid function capable of forming an amide function with this amine; if the active molecule is a protein with free thiol groups, then this functional group will be able to bear an activated acid function capable of forming a thioester function with this thiol. This functional group capable of forming a covalent bond with the primary and secondary amines, alcohols and thiols can therefore be selected from the group comprising the isothiocyanato, bromoacetamido, iodoacetamido, succinimido, pyridylthio, mercapto, maleimido, carboxyl groups and the ester derivatives (such as the N-hydroxy-succinimido, hydroxybenzotriazole and pentafluorophenyl groups), hydroxyl, aldehyde, amino, diazonium, tosyl, mesytylyl, trexyl, phosphodiester, phosphotriester.

By biologically active molecule is meant any molecule capable of taking part in a specific affinity reaction, in particular antigens, antibodies and fragments thereof, nucleic acids, peptides, polypeptides, hormones, lymphokines, growth factors, albumin, cytokines, enzymes, immunogenic modulators, receptors. By way of example, the antibodies or antibody fragments capable of reacting with the antigens associated with various pathologies, in particular those associated with diseases such as cancers (lymphomas, carcinomas, sarcomas, leukaemias, myelomas or tumours of the central nervous system), inflammatory diseases, cardiovascular diseases (thrombus, embolus, infarction, atherosclerotic plaque etc.), and infectious diseases may be mentioned.

According to the invention, the carrier bearing a group selected from the primary and secondary amines, alcohols and thiols can be presented in any form known to a person skilled in the art capable of being administered to a human being, in particular in the form of polymer beads or of metal or semiconductor nanoparticles. All these carriers are known to a person skilled in the art and are well described in the literature.

According to the invention, by salts is meant any salts known to a person skilled in the art, in particular pharmaceutically or biologically acceptable salts, such as for example salts with organic or inorganic bases, such as for example the salts of alkali metals, the alkaline-earth salts and the ammonium salts, water-soluble or water-insoluble. By way of example, the sodium, calcium and ammonium salts may be mentioned.

In an advantageous embodiment, a subject of the present invention is compounds of the following Formula (I):

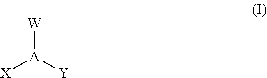

in which
A represents
   either a nitrogen atom,
   or a ring comprising from 3 to 6 carbon atoms, or an aromatic ring comprising from 5 to 10 members, said ring and said aromatic ring optionally comprising one or more heteroatoms selected from N, O and S,
W represents
   either a bromine atom, or an iodine atom,
   or an E-G-Q group with
      E selected from the group comprising an oxygen atom, a —C≡C— group, a (CH₂)ₘ, group, m being an integer comprised between 0 and 5, and a —CONH— group,
      G is selected from the group comprising
         i) —(CH₂)ₒ, o being an integer comprised between 0 and 5,
         ii) —(CH₂)ₙ—NH—, n being an integer comprised between 0 and 5,
         iii) —(CH₂)p-CO—NH—(CH₂)q-NH—, p and q being integers comprised between 0 and 5, and
         iv)

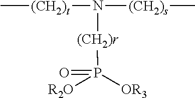

r, s and t being, each independently of one another, an integer comprised between 0 and 5, and R₂ and R₃ representing, each independently of one another, a hydrogen atom, a (C₁-C₄)alkyl group or a hydrolyzable group,
Q represents either a hydrogen atom, or an amine protecting group, or a functional group capable of forming a covalent bond with the primary and secondary amines, alcohols and thiols and
X and Y represent, independently of one another,
   either a hydrogen atom,
   or a

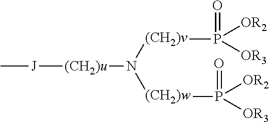

group, where u is an integer equal to 0 or 1, v and w, identical or different, are integers equal to 1 or 2, R₂ and R₃ represent, each independently of one another, a hydrogen atom, a (C₁-C₄)alkyl group or a hydrolyzable group selected from the group comprising the esters and the amides and J is either —CH₂, or is an aromatic ring comprising from 5 to 10 members and optionally one or more heteroatoms selected from N, O and S,
   or a

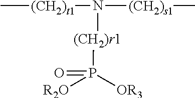

group, where r₁, s₁ and t₁ are, each independently of one another, an integer comprised between 1 and 2, and R₂ and R₃ represent, each independently of one another, a hydrogen atom, a (C₁-C₄)alkyl group or a hydrolyzable group,
provided that:
   X and Y, or
   X, W and Y,
are not simultaneously H,
and excluding the compounds of the following formula:

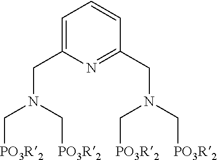

in which R′ represents H or Et,
and salts thereof.

In an advantageous embodiment, a subject of the present invention is compounds of the following Formula (I):

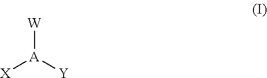

in which
   A is as defined above,
   W is as defined above with the exception of E, which is selected from the group comprising an oxygen atom, a —C≡C— group, and a —CONH— group,
   X and Y are as defined above, except that they cannot represent H simultaneously

In an advantageous embodiment of the invention, A represents an aromatic ring comprising from 5 to 10 members and optionally one or more heteroatoms selected from N, O and S, in particular A represents a pyridine, more advantageously a pyridine substituted in position 2 (position ortho to the nitrogen) by an X group, in position 4 (position para to the nitrogen) with a W group and in position 6 (position ortho to the nitrogen) with a Y group, X, Y and W being as defined in Formula (I).

In an advantageous embodiment, the compounds of Formula (I) in which A represents a pyridine substituted in position 2 (position ortho to the nitrogen) by an X group, in position 4 (position para to the nitrogen) with a W group and in position 6 (position ortho to the nitrogen) with a Y group are of the following Formula (I-a):

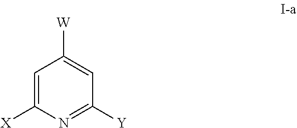

in which:
W represents
   either a bromine atom, or an iodine atom,
   or an E-G-Q group with
      E selected from the group comprising an oxygen atom, a —C≡C— group, a (CH₂)ₘ group, m being an integer comprised between 0 and 5, and a —CONH— group,
      G is selected from the group comprising
         i) —(CH₂)ₒ, o being an integer comprised between 0 and 5,
         ii) —(CH₂)ₙ—NH—, n being an integer comprised between 0 and 5,
         iii) —(CH₂)p-CO—NH—(CH₂)q-NH—, p and q being integers comprised between 0 and 5, and
         iv)

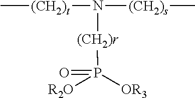

r, s and t being, each independently of one another, an integer comprised between 0 and 5, and R₂ and R₃ representing, each independently of one another, a hydrogen atom, a (C₁-C₄)alkyl group or a hydrolyzable group,
Q represents either a hydrogen atom, or an amine protecting group, or a functional group capable of forming a covalent bond with the primary and secondary amines, alcohols and thiols,
excluding the compounds in which W represents H, and
X and Y represent, independently of one another,
   either a hydrogen atom,
   or a

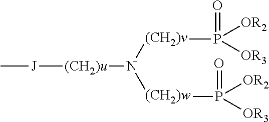

group, where u is an integer equal to 0 or 1, v and w, identical or different, are integers equal to 1 or 2, R₂ and R₃ represent, each independently of one another, a hydrogen atom, a (C₁-C₄)alkyl group or a hydrolyzable group selected from the group comprising the esters and the amides and J is either —CH₂, or is an aromatic ring comprising from 5 to 10 members and optionally one or more heteroatoms selected from N, O and S,
   or a

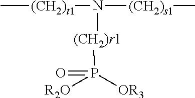

group, where r₁, s₁ and t₁ are, each independently of one another, an integer comprised between 1 and 2, and R₂ and R₃ represent, each independently of one another, a hydrogen atom, a (C₁-C₄)alkyl group or a hydrolyzable group,

provided that X and Y are not simultaneously H.

In an advantageous embodiment, the compounds of Formula (I) in which A represents a pyridine substituted in position 2 (position ortho to the nitrogen) with an X group, in position 4 (position para to the nitrogen) with a W group and in position 6 (position ortho to the nitrogen) with a Y group are of the following Formula (I-a):

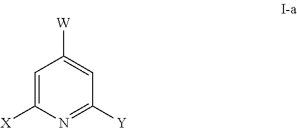

in which:
W represents
   either a bromine atom, or an iodine atom,
   or an E-G-Q group with
      E selected from the group comprising an oxygen atom, a —C≡C— group, and a —CONH— group,
      G is selected from the group comprising
         i) —(CH₂)ₒ, o being an integer comprised between 0 and 5,
         ii) —(CH₂)ₙ—NH—, n being an integer comprised between 0 and 5,
         iii) —(CH₂)p-CO—NH—(CH₂)q-NH—, p and q being integers comprised between 0 and 5, and
         iv)

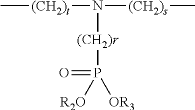

r, s and t being, each independently of one another, an integer comprised between 0 and 5, and R₂ and R₃ representing, each independently of one another, a hydrogen atom, a (C₁-C₄)alkyl group or a hydrolyzable group,
Q represents either a hydrogen atom, or an amine protecting group, or a functional group capable of forming a covalent bond with the primary and secondary amines, alcohols and thiols, and
X and Y represent, independently of one another,
   either a hydrogen atom,
   or a

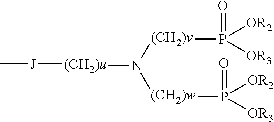

group, where u is an integer equal to 0 or 1, v and w, identical or different, are integers equal to 1 or 2, R₂ and R₃ represent, each independently of one another, a hydrogen atom, a (C₁-C₄)alkyl group or a hydrolyzable group selected from the group comprising the esters and the amides and J is either —CH₂, or is an aromatic ring comprising from 5 to 10 members and optionally one or more heteroatoms selected from N, O and S,
   or a

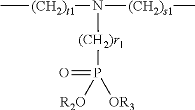

group, where r₁, s₁ and t₁ are, each independently of one another, an integer comprised between 1 and 2, and R₂ and R₃ represent, each independently of one another, a hydrogen atom, a (C₁-C₄)alkyl group or a hydrolyzable group,
provided that X and Y are not simultaneously H.

In an advantageous embodiment of the invention, in Formula (I), A represents an aromatic ring comprising from 5 to 10 members and optionally one or more heteroatoms selected from N, O and S, in particular A represents a pyridine, more advantageously a pyridine substituted in position 2 (position ortho to the nitrogen) with an X group, in position 4 (position para to the nitrogen) with a Y group and in position 6 (position ortho to the nitrogen) with a W group, the W, X and Y groups being as defined in said Formula (I).

In an advantageous embodiment, the compounds of Formula (I) in which A represents a pyridine substituted in position 2 (position ortho to the nitrogen) with an X group, in position 4 (position para to the nitrogen) with a Y group and in position 6 (position ortho to the nitrogen) with a W group are of the following Formula (I-b):

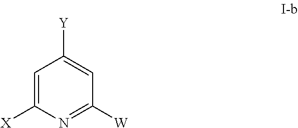

in which:
W, X and Y are as defined in Formula (I) excluding the compounds in which W and X represent H.

In an advantageous embodiment, the compounds of Formula (I) in which A represents a pyridine substituted in position 2 (position ortho to the nitrogen) with an X group, in position 4 (position para to the nitrogen) with a Y group and in position 6 (position ortho to the nitrogen) with a W group are of the following Formula (I-c):

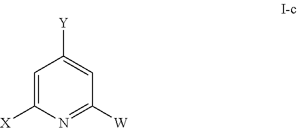

in which:
W represents an E-G-Q group with
   E representing a (CH₂)ₘ group, m being an integer comprised between 0 and 5
   G representing in the group comprising
      i)

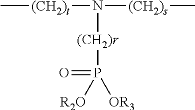

r, s and t being, each independently of one another, an integer comprised between 0 and 5, and R₂ and R₃ representing, each independently of one another, a hydrogen atom, a (C₁-C₄)alkyl group or a hydrolyzable group,
Q representing either a hydrogen atom, or an amine protecting group, or a functional group capable of forming a covalent bond with the primary and secondary amines, alcohols and thiols,
excluding the compounds in which W represents H, and
X is as defined in Formula (I) excluding H and
Y represents H.

In an advantageous embodiment, in the compounds of Formula (I-c), W represents an E-G-Q group as defined in Formula (I-c),
X represents:

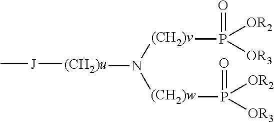

and Y represents H.

In an advantageous embodiment of the invention, the compounds of Formula (I) are those for which W is selected from the group comprising:
Br
—O—(CH₂)₃NHBoc,

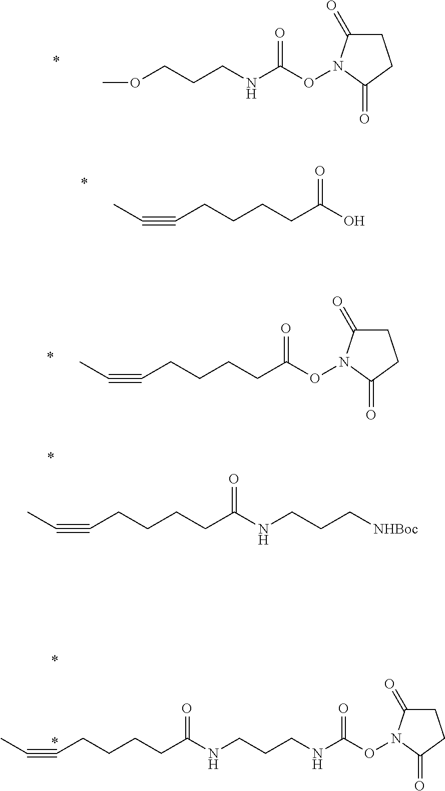

In another advantageous embodiment of the invention, the compounds of Formula (I) are those for which X and Y represent, each independently of one another, a group

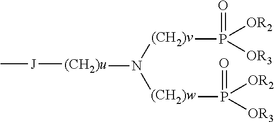

where J, R₂, R₃, u, v and w are as defined above.

In a particularly advantageous embodiment of the invention, the compounds of Formula (I) are those in which J represents a pyrazol-1-yl group or a pyridin-2-yl group.

In an even more advantageous embodiment of the invention, the compounds are those for which:
X represents a group

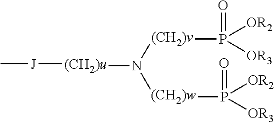

and Y represents a group

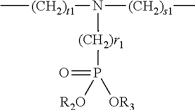

where J, R₂, R₃, u, v, w, t₁, r₁ and s₁ are as defined above.

Examples of compounds according to the invention correspond to the following formulae:

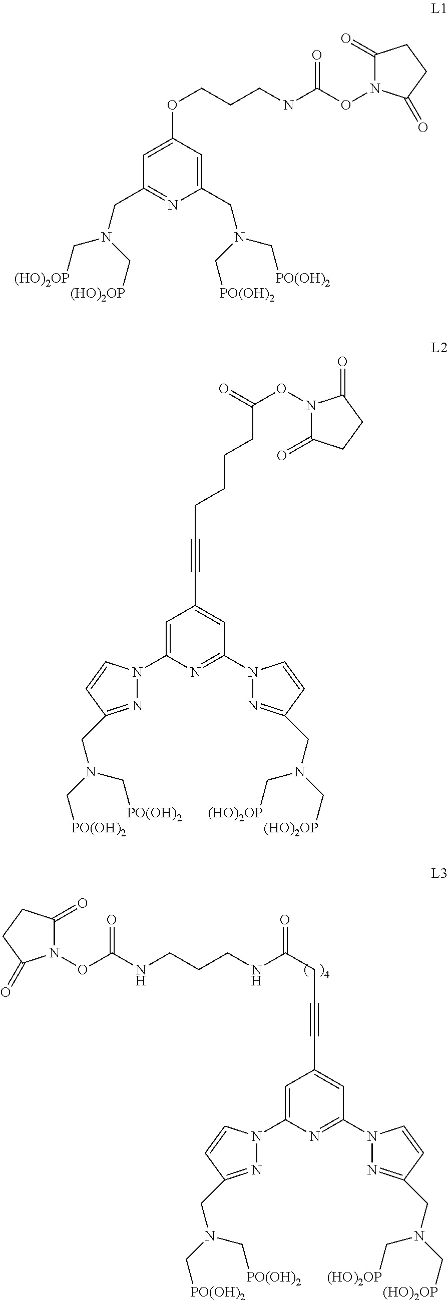

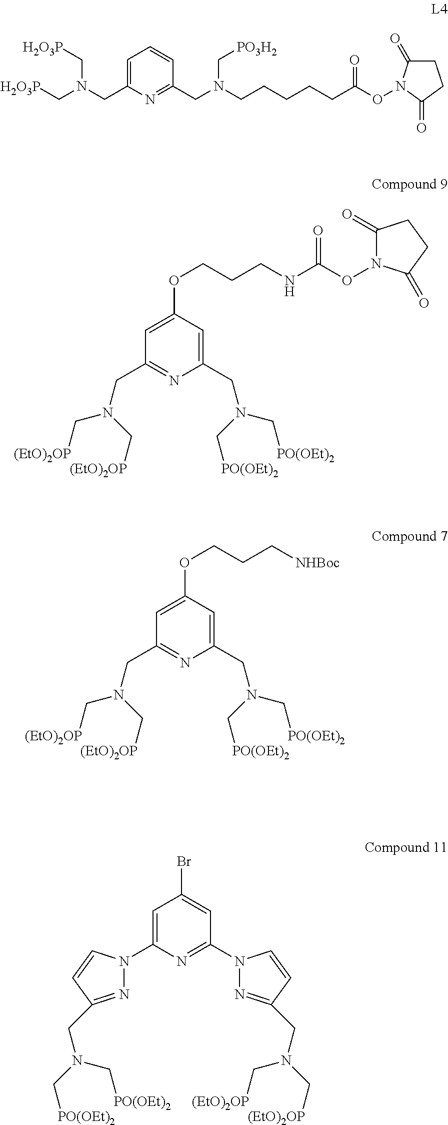

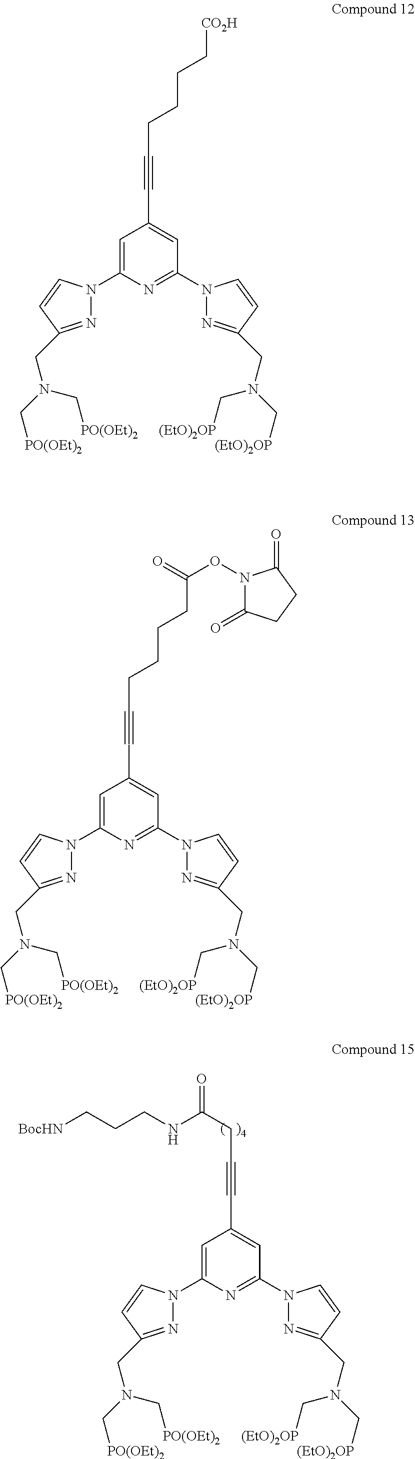

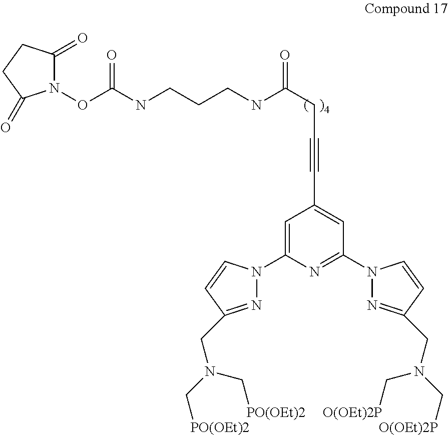

The compounds according to the invention can be synthesized by standard methods known to a person skilled in the art starting from compounds that are commercially available or the synthesis of which is described in the literature.

The key step in the synthesis of the compounds according to the invention is the step of selective deprotection of the phosphonic esters in the presence of an activated function. This step is carried out in the presence of trimethylsilyl bromide in a solvent such as dichloromethane or chloroform, in the presence of lutidine, followed by deprotection of the silylated esters with an alcohol, in particular methanol.

Thus, a further subject of the invention is a method for preparing the compounds of Formula (I) comprising a step of selective deprotection of the phosphonic esters in the presence of an activated function. This step comprises bringing a compound of Formula (I) bearing an activated function and at least one phosphonic ester function into contact with trimethylsilyl bromide in a solvent such as dichloromethane or chloroform, in the presence of lutidine, followed by deprotection of the silylated esters with an alcohol, in particular methanol.

The compounds according to the invention have, as central unit, a polysubstituted aromatic ring, in particular a polysubstituted pyridine nucleus, with polyaminophosphonate end groups and a side chain containing an activated chemical function. Owing to this innovative structure, these molecules are capable of chelating certain metals selectively and strongly, in particular by means of their phosphonate units. Compared with carboxylate groups, the phosphonate groups confer much greater stability for complexes formed with metals. This point is particularly important if the intended application requires very small quantities of cations, such as is the case in methods of imaging involving radioactive elements (PET, SPECT) or if the bifunctional chelating agents must be used for long periods (several days). In this case, the bifunctional chelating agents commonly used (functionalized 1,4,7,10-tetraazacyclododecane-1,4,7,10-tetraacetic acid (DOTA) for example) do not provide sufficient stability in vivo and the radioelements are released in the organism (Boswell A. et al. J. Med. Chem. 2004, 47, 1465).

A further subject of the present invention is therefore complexes comprising at least one metal ion coordinated with at least one compound of Formula (I).

By way of example, as metal ions, radiometals emitting gamma rays, radiometals emitting positrons, radiometals emitting alpha rays, radiometals emitting beta rays or radiometals emitting Auger electrons may be mentioned. It is also possible to use non-radioactive metals for their paramagnetic, fluorescence, or phosphorescence properties. By way of example, the lanthanides (europium, terbium, samarium, dysprosium, erbium, ytterbium, praseodymium and neodymium), iron, cobalt, nickel, copper (⁶⁴Cu, ⁶⁷Cu), zinc, arsenic, selenium, molybdenum, technetium, ruthenium, palladium, silver, cadmium, indium, antimony, rhenium, osmium, iridium, platinum, gold, mercury, thallium, lead, bismuth, polonium, gallium, zirconium, yttrium, scandium and astatine may be mentioned. All these metals can be used as they are or in the form of salts well known to a person skilled in the art.

They can be prepared by any technique known to a person skilled in the art, in particular by equimolar mixing of a compound of Formula (I) or a salt thereof with a metal salt, heating and cooling of the mixture and then neutralization to a pH comprised between 6 and 8 and finally recovery of the complex at ambient temperature.

In an advantageous embodiment, the metal of the complex formed with the ligand of Formula (I) is terbium.

Owing to the presence of a side chain containing an activated chemical function, the compounds according to the invention can also be grafted covalently on a biological target (proteins, antibodies, peptides) via said side chain containing the activated function.

Thus, a subject of the present invention is ligands comprising at least one complex formed with a compound of Formula (I) for which
W represents:
   an E-G-Q group with
      E selected from the group comprising an oxygen atom, a —C≡C— group, a (CH₂)ₘ group, m being an integer comprised between 0 and 5 and a group —CONH—,
      G is selected from the group comprising
         i) —(CH₂)ₒ, o being an integer comprised between 0 and 5,
         ii) —(CH₂)ₙ—NH—, n being an integer comprised between 0 and 5,
         iii) —(CH₂)p-CO—NH—(CH₂)q-NH—, p and q being integers comprised between 0 and 5, and
         iv)

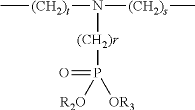

r, s and t being, each independently of one another, an integer comprised between 0 and 5, and R₂ and R₃ represent, each independently of one another, a hydrogen atom, a (C₁-C₄)alkyl group or a hydrolyzable group,
Q represents either a hydrogen atom, or an amine protecting group, or a functional group capable of forming a covalent bond with the primary and secondary amines, alcohols and thiols,
a target structure selected from the group comprising biologically active compounds or a carrier to form a conjugated system.

In an advantageous embodiment, the conjugated system is constituted by:
the ligand L1 to which an antibody is bound, in particular the B28.13 antibody directed against tenascin, or
the ligand L2 to which:
   an antibody is bound, in particular the dreg55 antibody having very strong affinity for L-selectin or the dreg200 antibody, a mouse antibody directed against L-selectin, or the PSS233 antibody, an anti “prostate specific antigen” (PSA) antibody, or the PSR222 antibody, an anti “prostate specific antigen” (PSA) antibody, or the EgB4 antibody, antibody fragment constituted by a single variable domain and directed against the epidermal growth factor, or the EgA1 antibody, antibody fragment constituted by a single variable domain and directed against the epidermal growth factor, or
   a protein is bound, in particular L-selectin.

The B28.13 antibody can be obtained according to S. Wagner et al. (Early osteoarthritic changes of human femoral head cartilage subsequent to femoro-acetabular impingement” Osteoarthritis and Cartilage, 2003, 11, 508).

The dreg55 and dreg200 antibodies can be obtained according to Man Sung Co et al. (Properties and pharmacokinetics of two humanized antibodies specific for L-selectin, Immunotechnology 4 (1999) 253-266).

The PSS233, PSR222 antibodies and the EgB4 or EggA1 fragments can be obtained from THERMO FISHER SCIENTIFIC CD NIMES CEZANNE, 280 allée Graham Bell, Parc Scientifique Georges Besse, 30035 Nîmes Cedex 1 France).

In an advantageous embodiment, the conjugated system is constituted by:
the ligand L1 to which an antibody is bound, in particular the B28.13 antibody directed against tenascin, or
the ligand L2 to which:
   an antibody is bound, in particular the antibody bound is the dreg55 antibody having very strong affinity for L-selectin or the dreg 200 antibody, a mouse antibody directed against L-selectin, is bound, or
   a protein is bound, in particular L-selectin.

A further subject of the present invention is ligands comprising at least one complex formed with a compound of Formula (I) for which
W represents:
   an E-G-Q group with
      E selected from the group comprising an oxygen atom, a —C≡C— group, a (CH₂)ₘ group, m being an integer comprised between 0 and 5 and a —CONH— group,
      G is selected from the group comprising
         i) —(CH₂)ₒ, o being an integer comprised between 0 and 5,
         ii) —(CH₂)ₙ—NH—, n being an integer comprised between 0 and 5,
         iii) —(CH₂)p-CO—NH—(CH₂)q-NH—, p and q being integers comprised between 0 and 5, and
         iv)

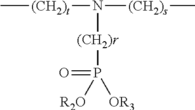

r, s and t being, each independently of one another, an integer comprised between 0 and 5, and R₂ and R₃ represent, each independently of one another, a hydrogen atom, a (C₁-C₄)alkyl group or a hydrolyzable group,
Q represents either a hydrogen atom, or an amine protecting group, or a functional group capable of forming a covalent bond with the primary and secondary amines, alcohols and thiols,
a metal coordinated to said compound of Formula (I) and a target structure selected from the group comprising biologically active compounds or a carrier to form a complexed conjugated system.

These complexes can be prepared by any technique known to a person skilled in the art, in particular by equimolar mixing of the compound of Formula (I) and of a water-soluble metal salt in an aqueous medium followed by isolation of the complex by precipitation or evaporation to dryness or chromatography.

A further subject of the present invention is a conjugated system comprising a bifunctional agent of Formula (I) in which
W represents:
   an E-G-Q group with
      E selected from the group comprising an oxygen atom, a —C≡C— group, a (CH₂)ₘ group, m being an integer comprised between 0 and 5 and a —CONH— group,
      G is selected from the group comprising
         i) —(CH₂)ₒ, o being an integer comprised between 0 and 5,
         ii) —(CH₂)ₙ—NH—, n being an integer comprised between 0 and 5,
         iii) —(CH₂)p-CO—NH—(CH₂)q-NH—, p and q being integers comprised between 0 and 5, and
         iv)

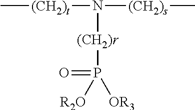

r, s and t being, each independently of one another, an integer comprised between 0 and 5, and R₂ and R₃ represent, each independently of one another, a hydrogen atom, a (C₁-C₄)alkyl group or a hydrolyzable group,
Q represents either a hydrogen atom, or an amine protecting group, or a functional group capable of forming a covalent bond with the primary and secondary amines, alcohols and thiols of a biologically active molecule or of a carrier,
and a biologically active compound or a carrier.

These conjugated systems can be prepared by any technique known to a person skilled in the art, in particular by a coupling reaction of the bifunctional agent of Formula (I) with the biologically active compound or carrier, in a buffered aqueous medium, at a temperature between 4 and 40° C., followed by purification by chromatography.

In a particularly advantageous embodiment of the invention, the complex according to the invention or the conjugated system according to the invention is formed with a bifunctional ligand selected from the group comprising L1, L2, L3 and L4 as defined above.

Although complexes having phosphonated functions have been described extensively in the literature, this is the first time that the introduction of an activation function for covalently binding such structures to biological compounds has been described. The molecules described to date as having a grafting function are derivatives the complexing functions of which are generally carboxylates, for which the complexation of the cations is much weaker, especially when the pH decreases.

In an advantageous embodiment, the complexed conjugated system is constituted by the conjugated systems defined above complexed to a metal, in particular terbium.

A further subject of the present invention is a diagnostic agent comprising at least one compound of Formula (I) according to the invention or a complex according to the invention.

This agent can be used in any imaging technique known to a person skilled in the art, in particular in nuclear magnetic resonance (NMR), in magnetic resonance imaging (MRI), in radiography with X-rays, in positron emission tomography (PET), in single-photon emission tomography (SPET or SPECT), in radiology, in luminescence spectroscopy.

According to the invention, the diagnostic agent can be used as an imaging agent in nuclear magnetic resonance with the following metal ions: Eu, Yb, Gd, Dy, Tb, Ho, Er or Fe, as an X-ray imaging agent with the following metal ions: Bi, Pb or Os, as an imaging agent in radiology with the following metal ions: Co, Cu, Ga, Ge, Sr, Y, Tc, In, Sm, Gd, Tb, Yb, Re, Zr or Ir or by luminescence spectroscopy with the following metal ions: Eu, Tb, Dy, Sm, Yb.

According to the present invention, the diagnostic agent can be presented in any pharmaceutically acceptable form prepared by any technique known to a person skilled in the art, in particular by mixing a quantity of complex with any pharmaceutically acceptable additive known to a person skilled in the art. Advantageously it is presented in the form of an injectable solution prepared by dissolving the complex in a physiologically acceptable aqueous solvent.

The doses used will be adapted according to the type of imaging used and this adaptation is within the capability of a person skilled in the art. For diagnosis by NMR, the dose of metal is generally from 0.0001 to 10 mmol/kg, advantageously from 0.005 to 0.5 mmol/kg. For diagnosis with X-rays, the dose of metal ion is generally from 0.01 to 20 mmol/kg, advantageously from 0.1 to 10 mmol/kg. Moreover, in radiology, the dose of radioactivity is from 370-18500 MBq. The imaging agent is generally administered by parenteral route, in particular by intravenous route but in certain cases it can be administered orally, or by intraarterial route.

According to the invention, the compounds of Formula (I) and the complexes as defined above can be used for detecting diseases such as lymphomas, carcinomas, sarcomas, leukaemias, myelomas or tumours of the central nervous system, inflammatory diseases, cardiovascular diseases (thrombus, embolus, infarction, atherosclerotic plaque etc.), and infectious diseases.

A subject of the present invention is therefore a diagnostic agent as defined above for use in the detection of the aforementioned diseases.

A further subject of the present invention is a method for detection of a disease that produces or is associated with a marker or with a receptor, said method comprising the administration, to a human subject who has said disease, of a detectable quantity of a complex as defined above and comprising a biologically active molecule specific to said marker or said receptor. According to the invention, the compounds of Formula (I) and the complexes as defined above can also be used for treating diseases such as lymphomas, carcinomas, sarcomas, leukaemias, myelomas or tumours of the central nervous system, inflammatory diseases, cardiovascular diseases (thrombus, embolus, infarction, atherosclerotic plaque, etc.), and infectious diseases.

A subject of the present invention is therefore a complex as defined above for use in the treatment of the aforementioned diseases. In this context it can be presented in any pharmaceutical form known to a person skilled in the art in combination with any pharmaceutically acceptable excipient. Advantageously it is presented in the form of an injectable solution prepared by dissolving the complex in a physiologically acceptable aqueous solvent.

A further subject of the present invention is a method for treatment of a disease that produces or is associated with a marker or a receptor, said method comprising the administration, to a human subject who has said disease, of a detectable quantity of a complex as defined above and comprising a biologically active molecule specific to said marker or said receptor. The complexes according to the invention are prepared by the techniques known to a person skilled in the art.

A further subject of the present invention is a kit comprising:
(1) a bifunctional ligand as defined above.

In an advantageous embodiment, a further subject of the present invention is a kit comprising:
(1) a bifunctional ligand as defined above,
(2) a solution of a salt or of a chelate of a metallic radionuclide and
(3) if necessary, instructions for use for reaction with a prescription for reacting (1) and (2).

A further subject of the present invention is a kit for preparing a diagnostic agent comprising:
(1) a bifunctional ligand as defined above and
(2) if necessary, instructions for use for reaction with a prescription for its use

A further subject of the present invention is a kit comprising:
(1) a bifunctional ligand as defined above,
(2) a solution of a salt or of a chelate of a metallic radionuclide and
(3) instructions for use, with a prescription for reacting (1) and (2) with a biologically active compound or an inert carrier.

The compounds of Formula (I) in which
W represents
   either a bromine atom, or an iodine atom,
   or an E-G-Q group with
      E selected from the group comprising an oxygen atom, a —C≡C— group, a (CH₂), group, m being an integer comprised between 0 and 5 and a —CONH— group,
      G is selected from the group comprising
         i) —(CH₂)ₒ, o being an integer comprised between 0 and 5,
         ii) —(CH₂)ₙ—NH—, n being an integer comprised between 0 and 5,
         iii) —(CH₂)p-CO—NH—(CH₂)q-NH—, p and q being integers comprised between 0 and 5, and
         iv)

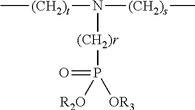

r, s and t being, each independently of one another, an integer comprised between 0 and 5, and R₂ and R₃ represent, each independently of one another, a hydrogen atom, a (C₁-C₄)alkyl group or a hydrolyzable group,
Q represents either a hydrogen atom, or an amine protecting group,
X and Y represent, independently of one another,
   either a hydrogen atom,
   or a

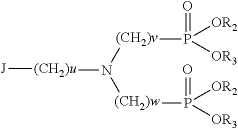

group, where u is an integer equal to 0 or 1, v and w, identical or different, are integers equal to 1 or 2, R₂ and R₃ represent, each independently of one another, a (C₁-C₄)alkyl group and J is either —CH₂, or is an aromatic ring comprising from 5 to 10 members and optionally one or more heteroatoms selected from N, O and S,
   or a

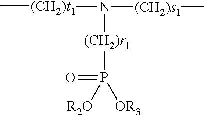

group, where r₁, s₁ and t₁ are, each independently of one another, an integer comprised between 1 and 2, and R₂ and R₃ represent, each independently of one another, a (C₁-C₄)alkyl group or a hydrolyzable group,
provided that X, W and Y are not simultaneously H,
as intermediates in the synthesis of the compounds of Formula (I) in which W represents
   an E-G-Q group with
      E selected from the group comprising an oxygen atom, a —C≡C— group, a (CH₂)ₘ group, m being an integer comprised between 0 and 5 and a —CONH— group,
      G is selected from the group comprising
         i) —(CH₂)ₒ, o being an integer comprised between 0 and 5,
         ii) —(CH₂)ₙ—NH—, n being an integer comprised between 0 and 5,
         iii) —(CH₂)p-CO—NH—(CH₂)q-NH—, p and q being integers comprised between 0 and 5, and
         iv)

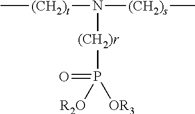

r, s and t being, each independently of one another, an integer comprised between 0 and 5, and R₂ and R₃ represent, each independently of one another, a hydrogen atom, a (C₁-C₄)alkyl group or a hydrolyzable group,
Q represents either a hydrogen atom, or a functional group capable of forming a covalent bond with the primary and secondary amines, alcohols and thiols
X and Y represent, independently of one another,
   either a hydrogen atom,
   or a

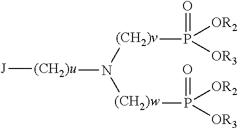

group, where u is an integer equal to 0 or 1, v and w, identical or different, are integers equal to 1 or 2, R₂ and R₃ represent, each independently of one another, a hydrogen atom and J is either —CH₂, or is an aromatic ring comprising from 5 to 10 members and optionally one or more heteroatoms selected from N, O and S,
   or a

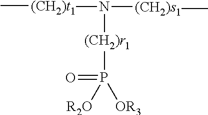

group, where r₁, s₁ and t₁ are, each independently of one another, an integer comprised between 1 and 2, and R₂ and R₃ represent, each independently of one another, a hydrogen atom,
provided that X, W and Y are not simultaneously H.

In an advantageous embodiment, in the compounds defined above:
X and Y, or
X, W and Y,
are not simultaneously H,
and the compounds of the following formula:

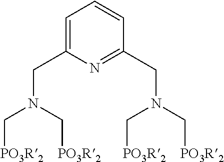

in which R′ represents H or Et are excluded.

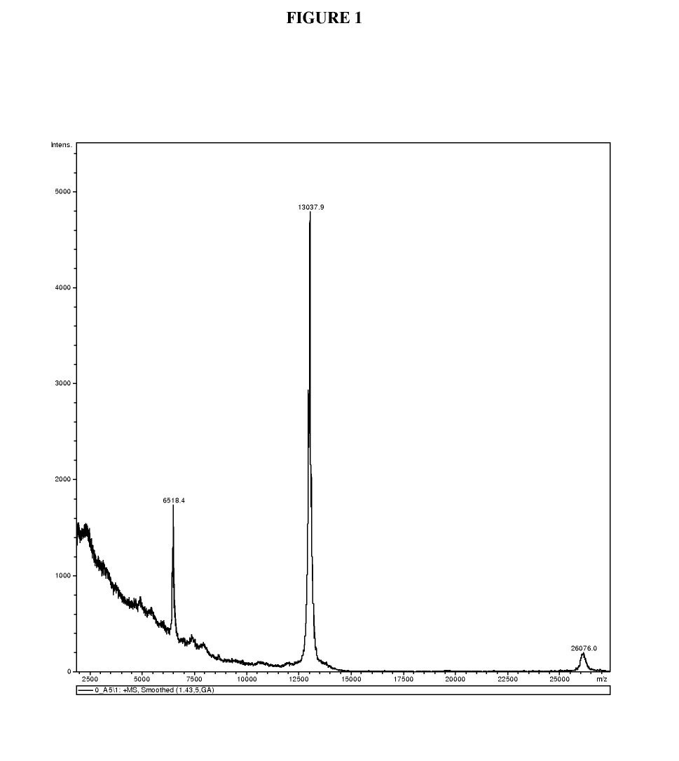

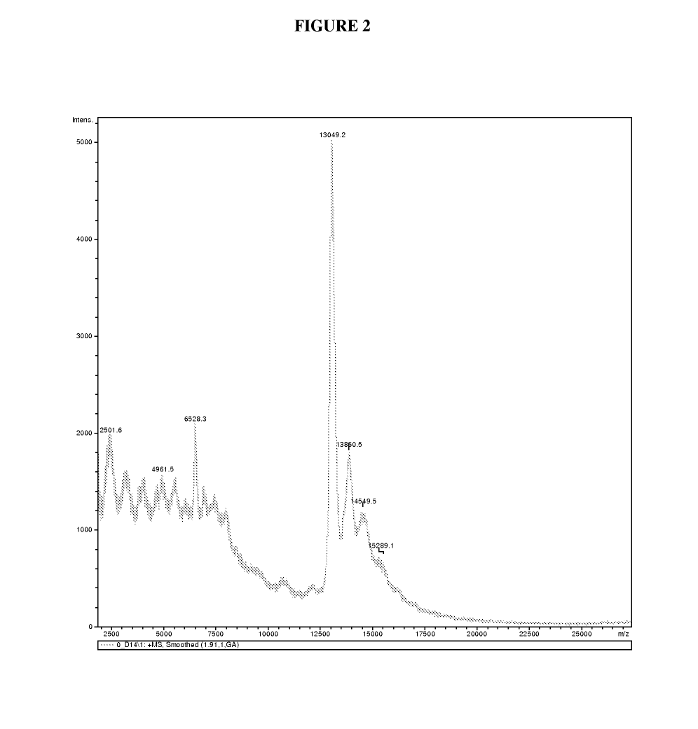

FIGS. 1 and 2 and the following examples illustrate the invention.

FIG. 1 shows the mass spectrum of the reference streptavidin in mQ water obtained by MALDI-MS mass spectrometry according to Example 5.

FIG. 2 shows the mass spectrum of streptavidin labelled with ligand L₂ obtained by MALDI-MS mass spectrometry according to Example 5.

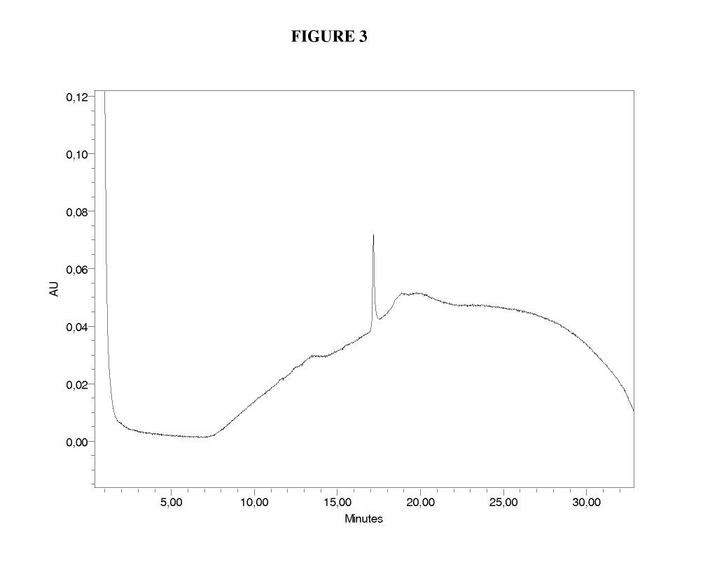

FIG. 3 shows the chromatogram obtained by HPLC showing the injection of 0.2 μg of the B28.13 antibody.

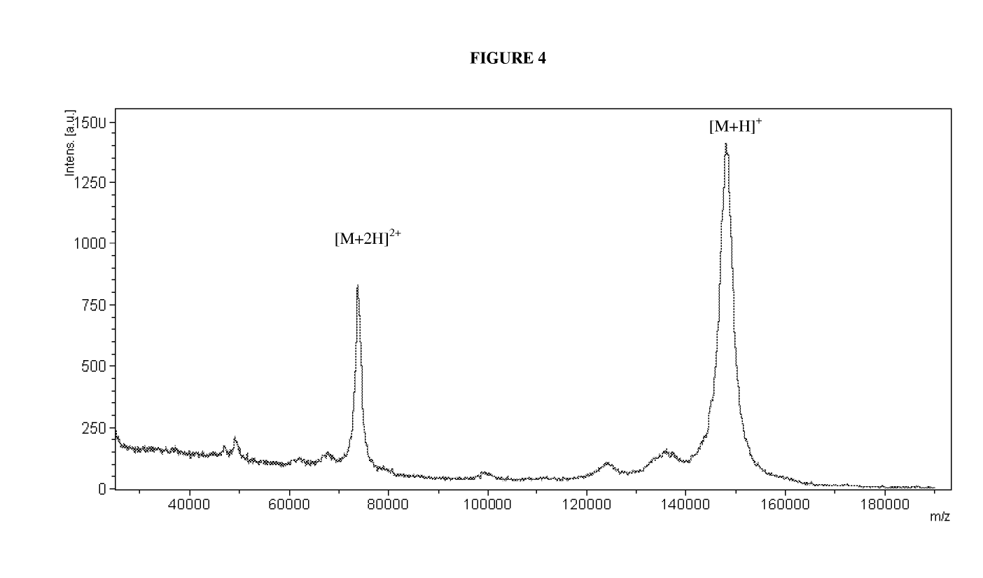

FIG. 4 shows the MALDI-TOF spectrum of the B28.13 antibody.

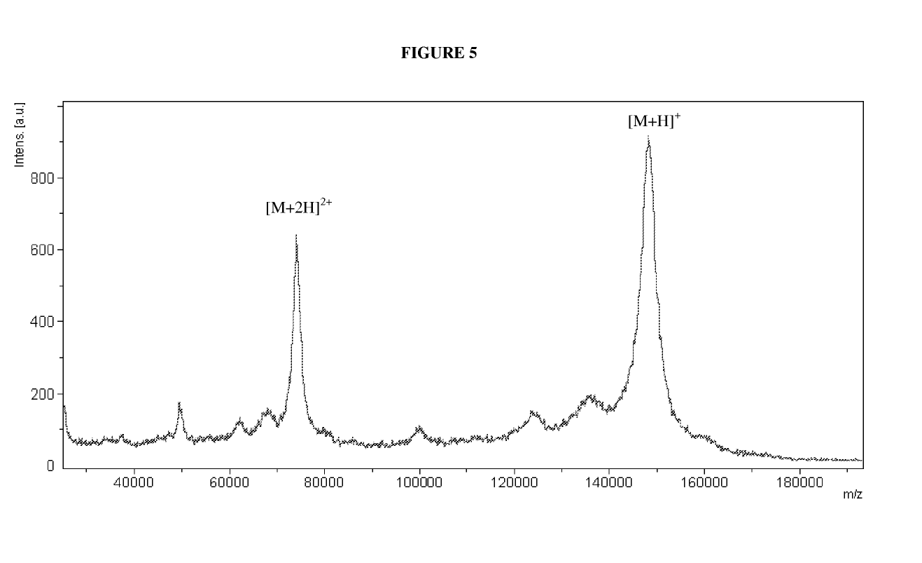

FIG. 5 shows the MALDI-TOF spectrum of the labelled B28.13 antibody.

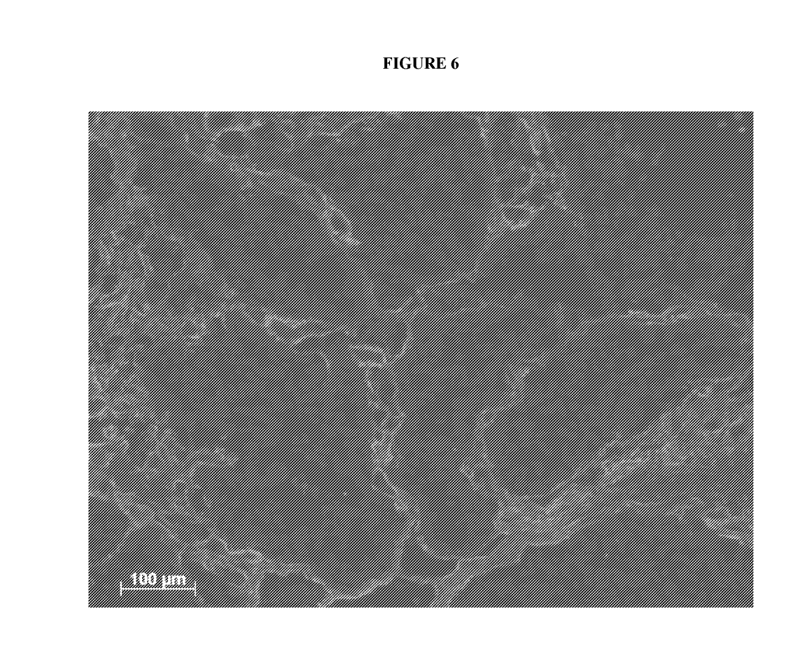

FIG. 6 shows the fluorescence microscopy image, showing recognition of the extracellular tenascin that develops around the colon tumours with the labelled B28.13 antibody.

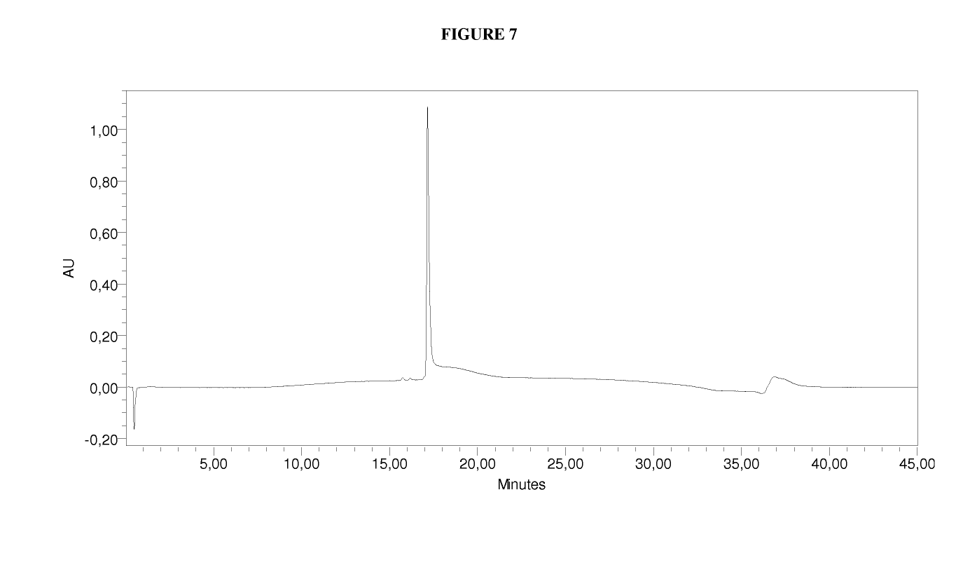

FIG. 7 shows the chromatogram showing injection of 2 μg of dreg55 (injection of 20 μL of a solution of dreg55 at 0.1 mg/mL).

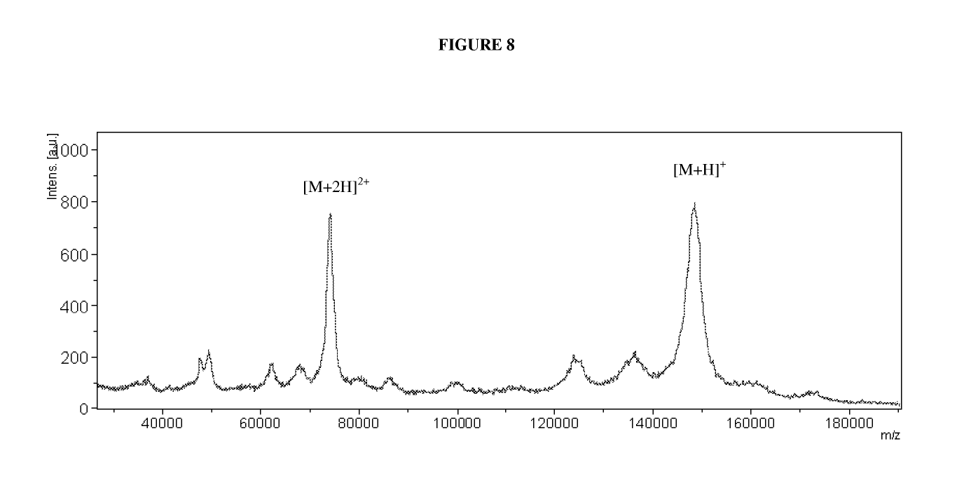

FIG. 8 shows the MALDI-TOF spectrum of the dreg55 antibody.

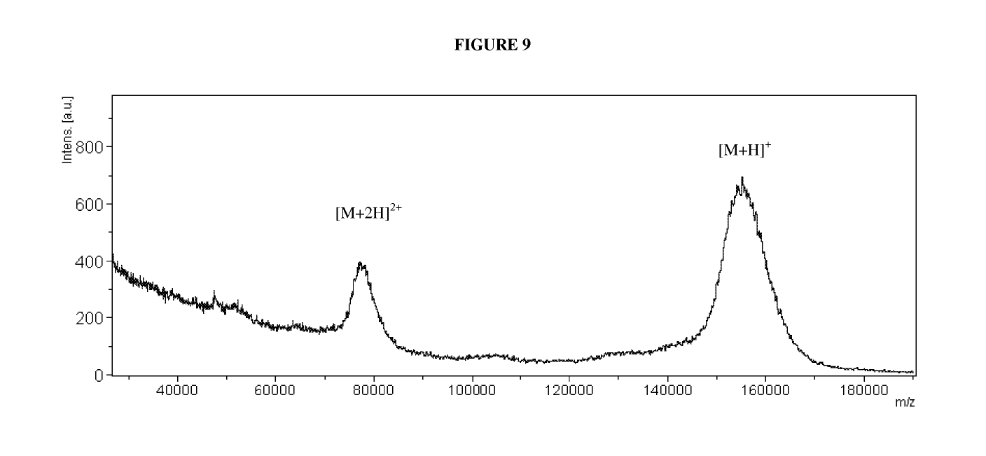

FIG. 9 shows the MALDI-TOF spectrum of the labelled dreg55 antibody.

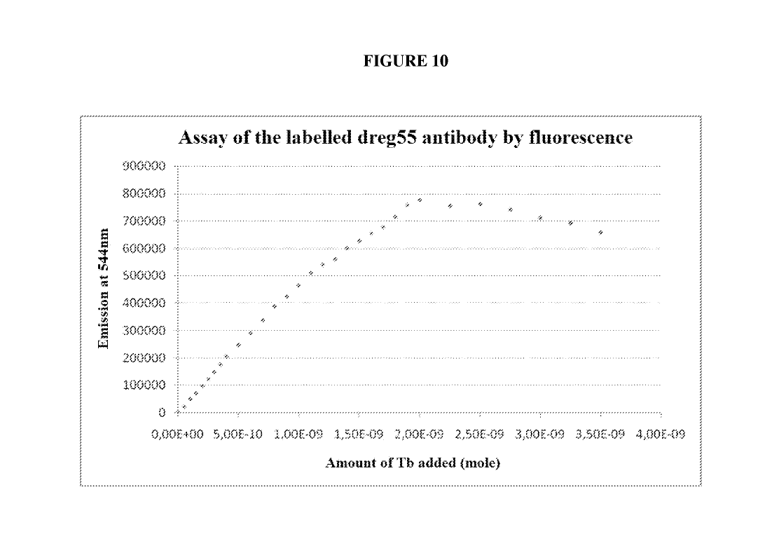

FIG. 10 shows the fluorescence assay of the labelled dreg55 antibody.

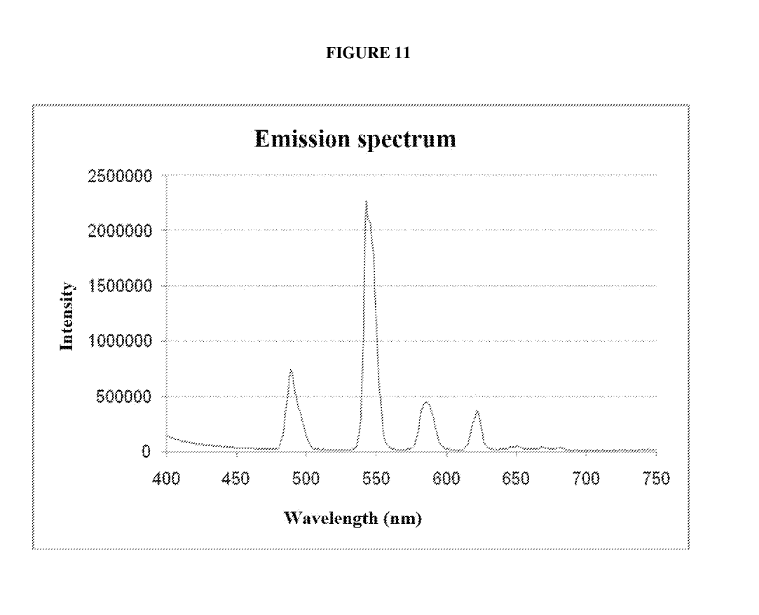

FIG. 11 shows the emission spectrum of the TbL₂-labelled dreg55 antibody (λ_{exc.}=328 nm).

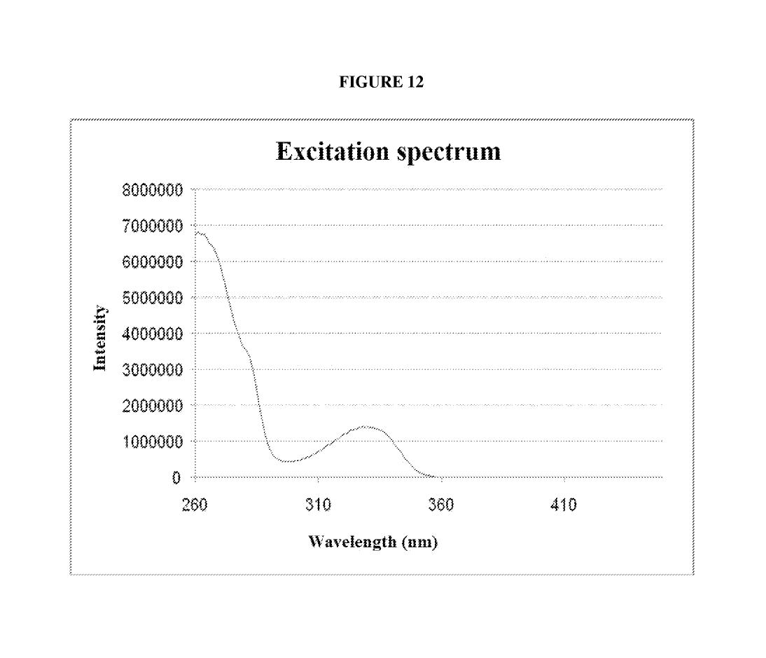

FIG. 12 shows the excitation spectrum of the labelled dreg55 antibody.

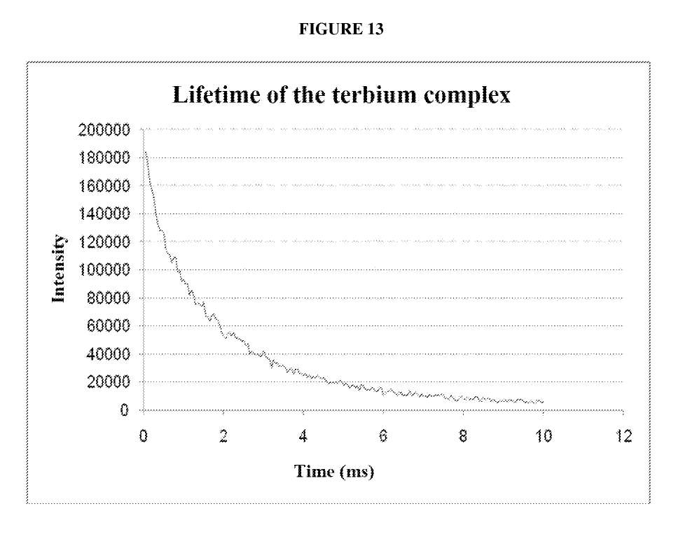

FIG. 13 shows the decrease in luminescence of the terbium complex.

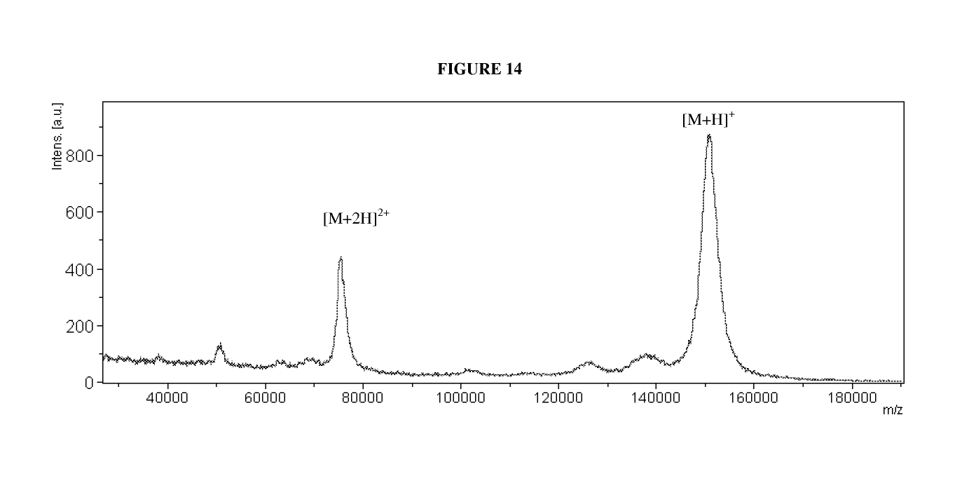

FIG. 14 shows the MALDI-TOF spectrum of the dreg200 antibody.

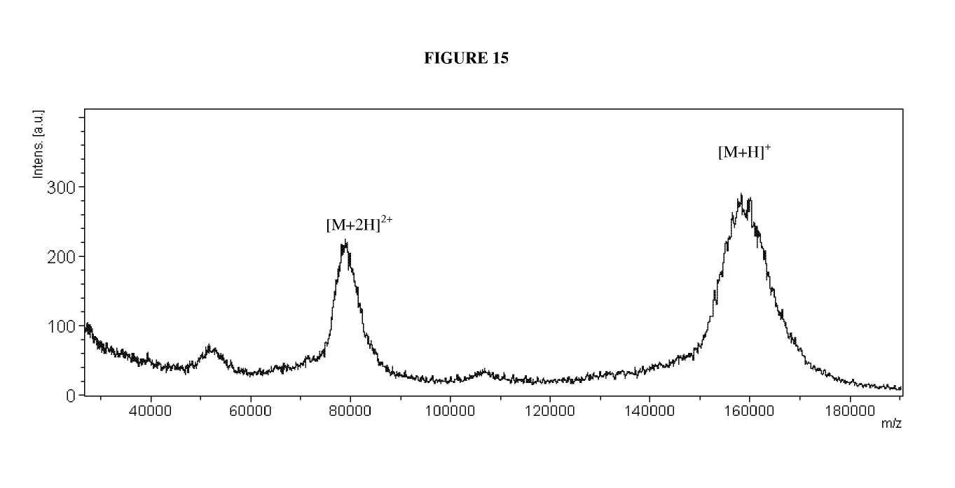

FIG. 15 shows the MALDI-TOF spectrum of the labelled dreg200 antibody.

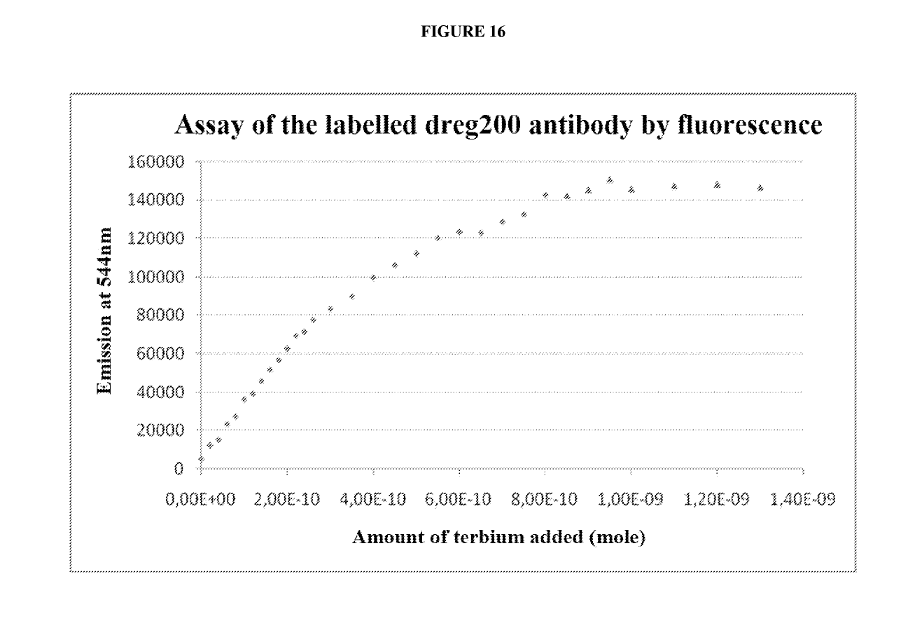

FIG. 16 shows the fluorescence assay of the labelled dreg200 antibody.

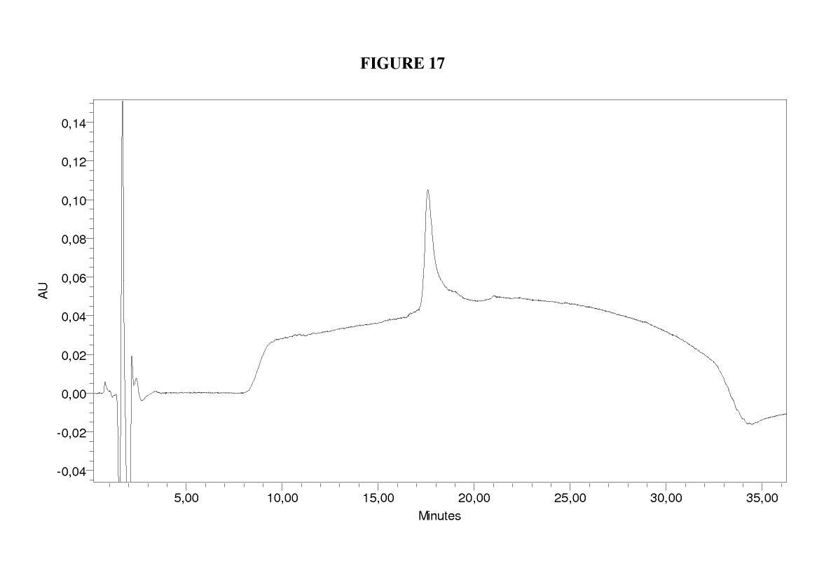

FIG. 17 shows the chromatogram showing injection of 0.75 μg of L-selectin (injection of 20 μL of a solution of L-selectin at 0.037 mg/mL).

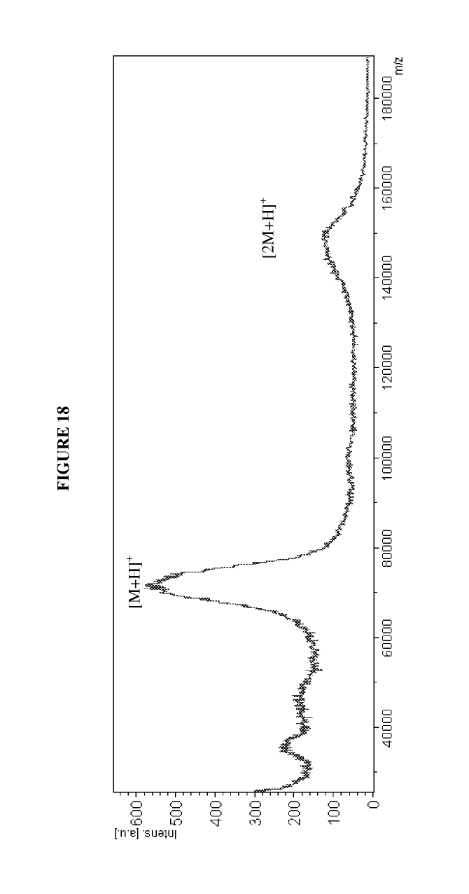

FIG. 18 shows the MALDI-TOF spectrum of L-selectin.

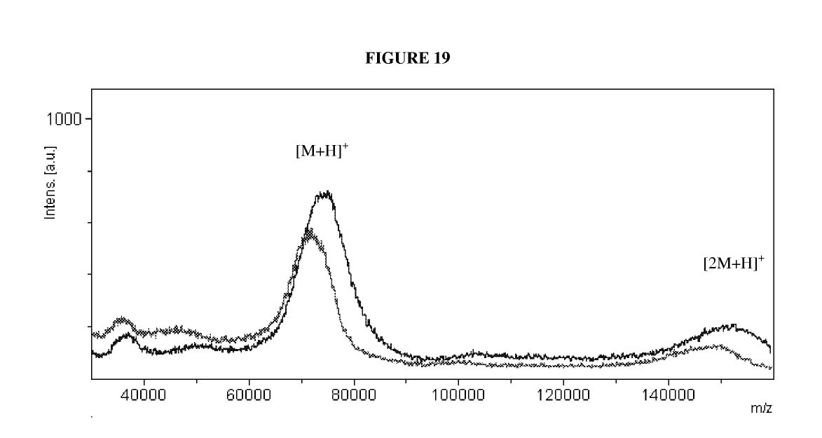

FIG. 19 shows the superimposition of the MALDI-TOF spectra of L-selectin (light grey) and labelled L-selectin (black).

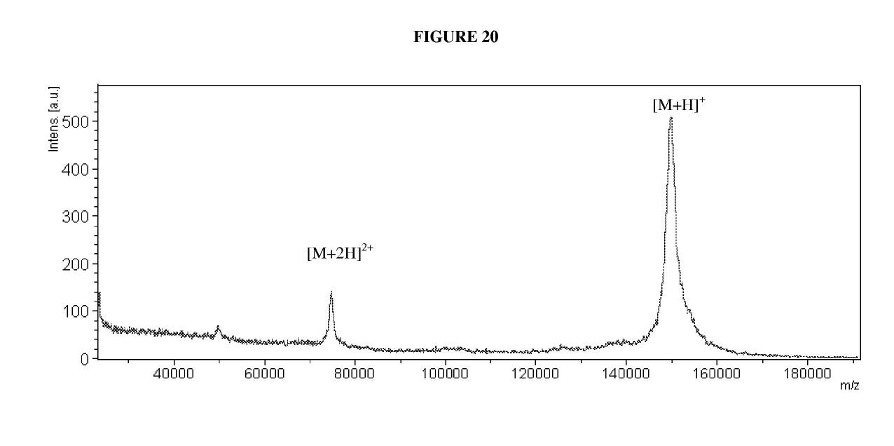

FIG. 20 shows the MALDI-TOF spectrum of the PSS233 antibody.

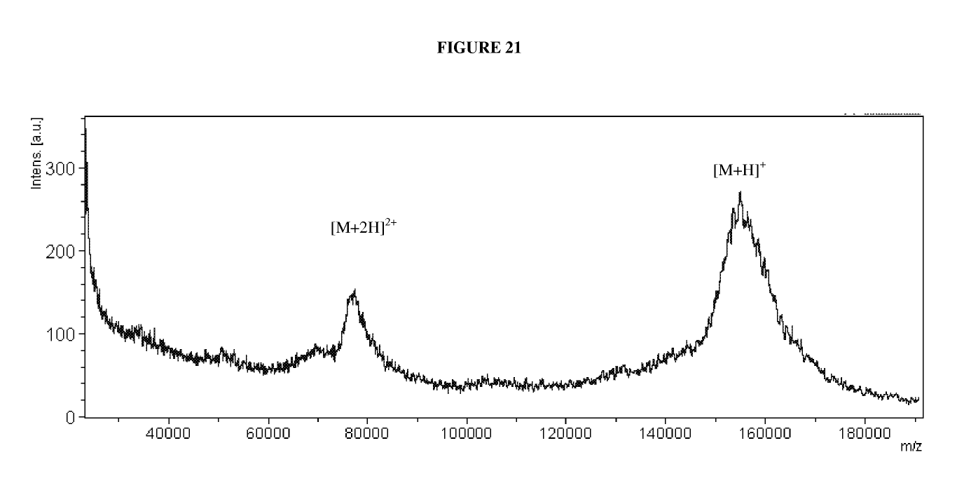

FIG. 21 shows the MALDI-TOF spectrum of the labelled PSS233 antibody.

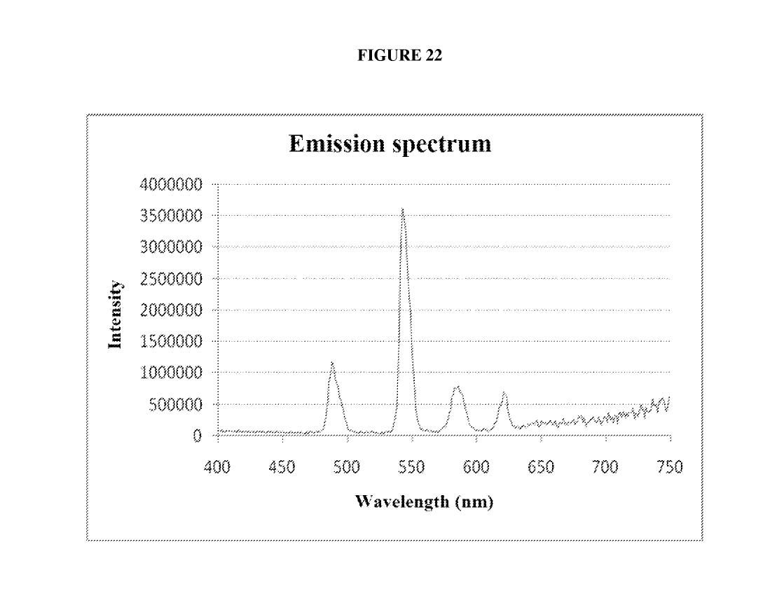

FIG. 22 shows the emission spectrum of the labelled PSS233 antibody (λ_{exc.}=328 nm).

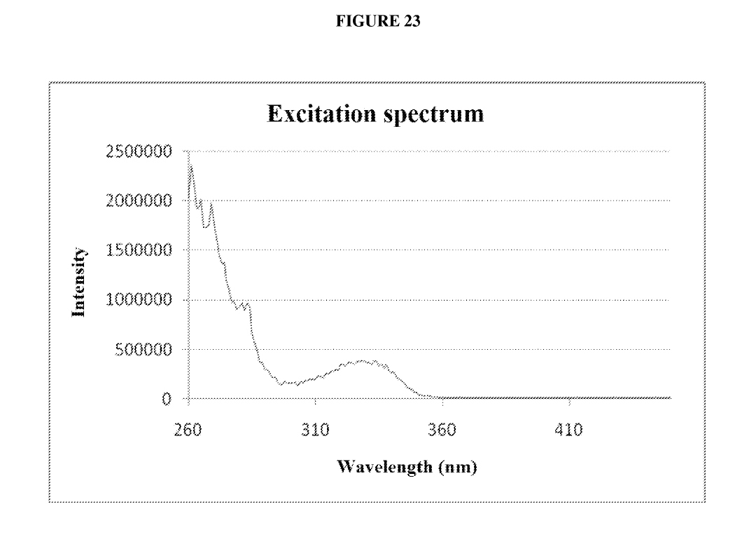

FIG. 23 shows the excitation spectrum of the labelled PSS233 antibody.

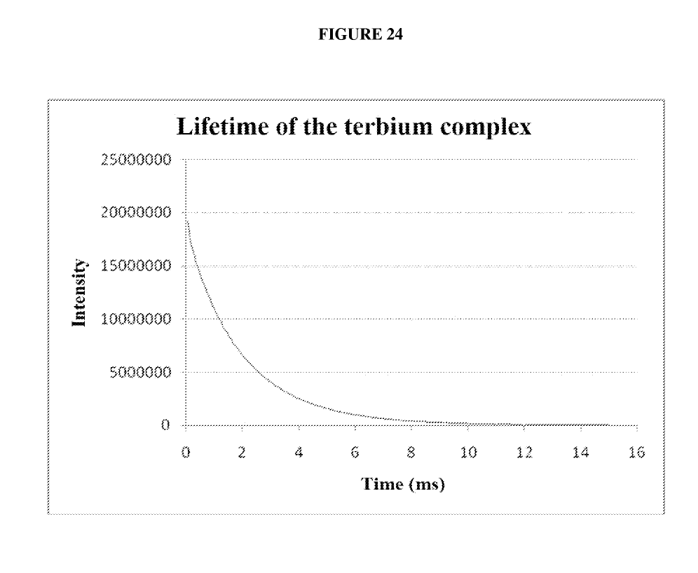

FIG. 24 shows the decrease in luminescence of the terbium complex.

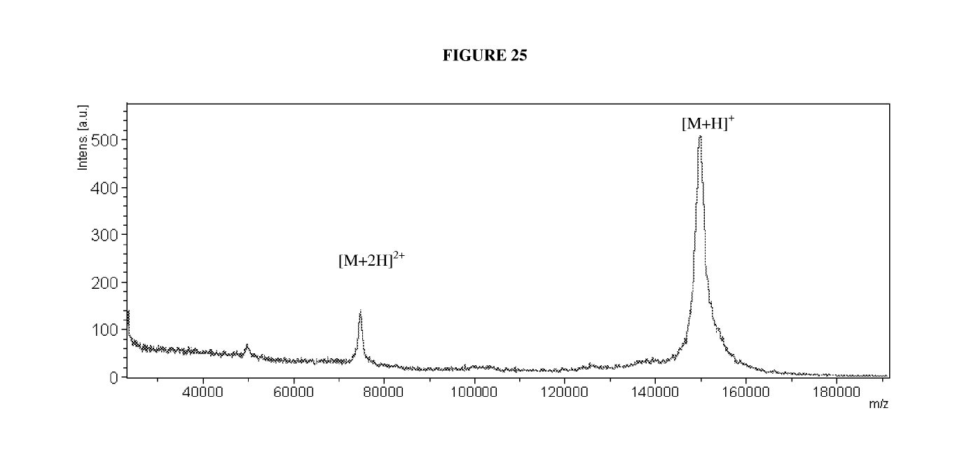

FIG. 25 shows the MALDI-TOF spectrum of the PSR222 antibody.

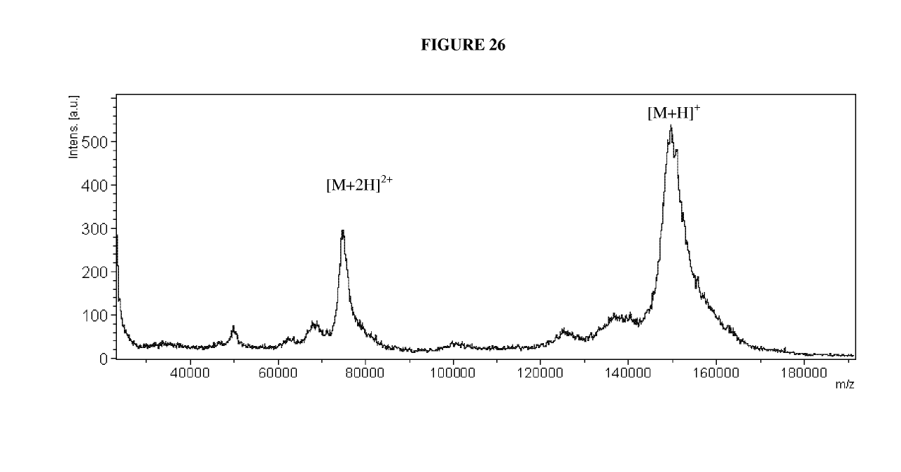

FIG. 26 shows the MALDI-TOF spectrum of the labelled PSR222 antibody.

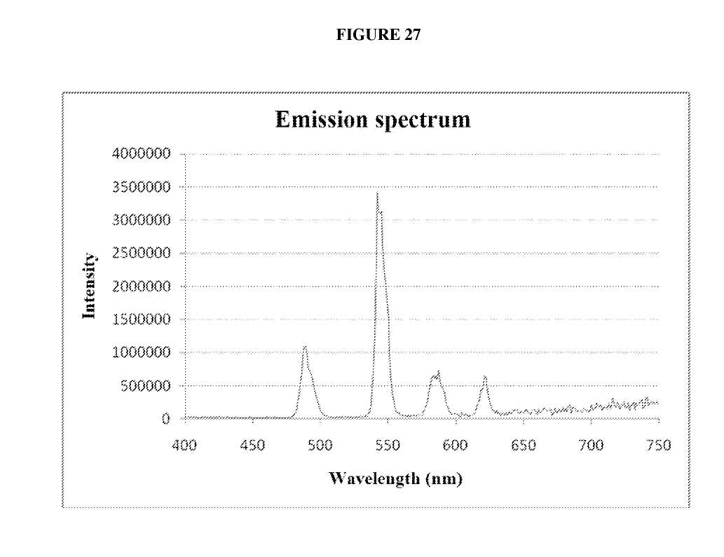

FIG. 27 shows the emission spectrum of the labelled PSR222 antibody (λ_{exc.}=328 nm).

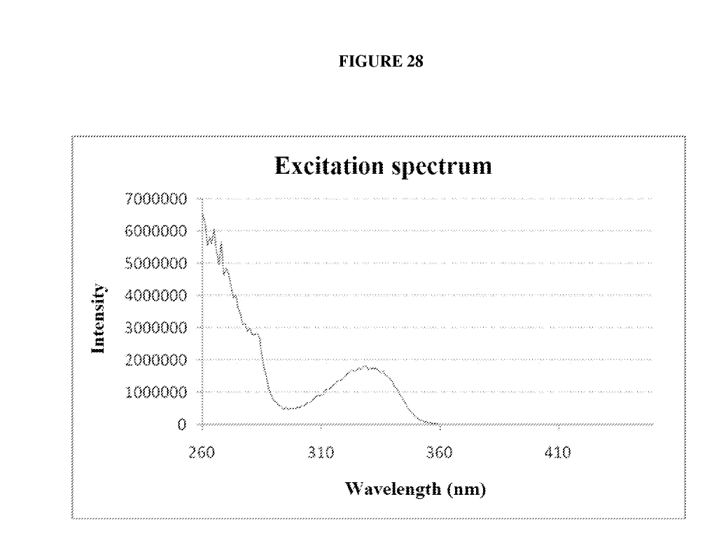

FIG. 28 shows the excitation spectrum of the labelled PSR222 antibody.

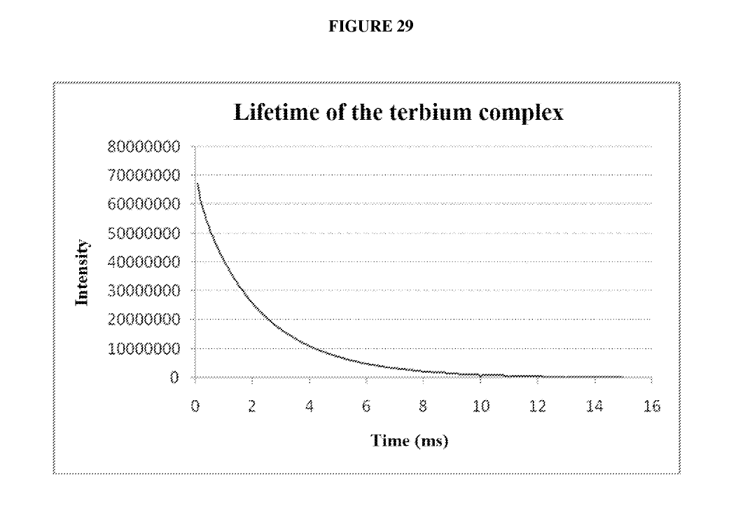

FIG. 29 shows the decrease in luminescence of the terbium complex.

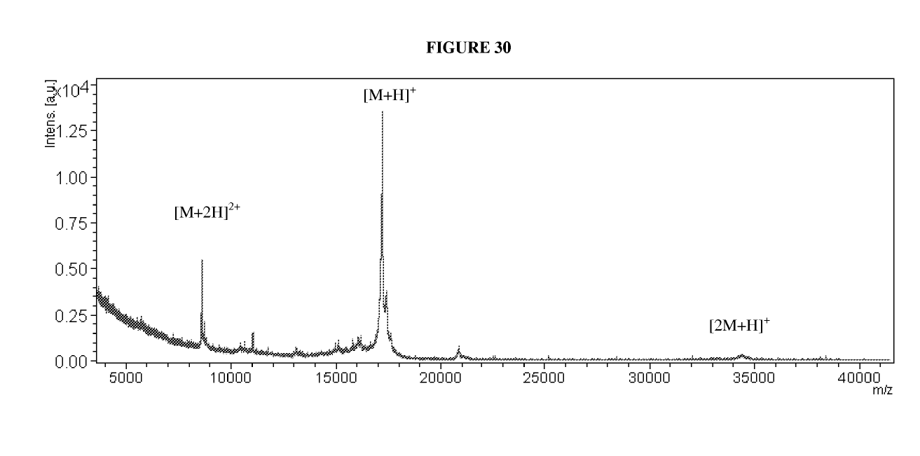

FIG. 30 shows the MALDI-TOF spectrum of the EgB4 antibody fragment.

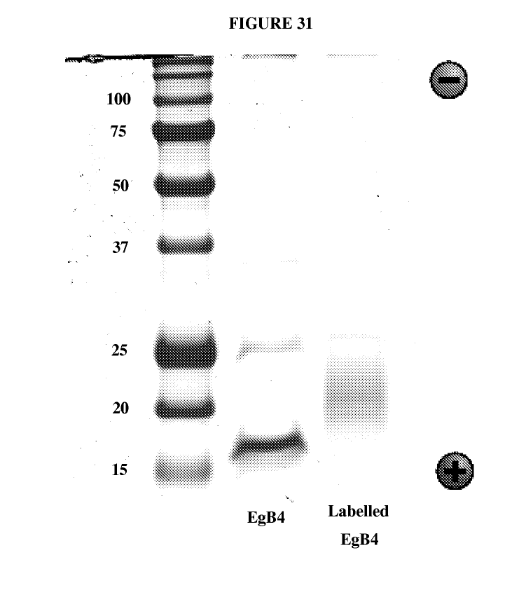

FIG. 31 shows tricine-gel SDS-PAGE of the EgB4 antibody fragment and labelled EgB4 antibody fragment.

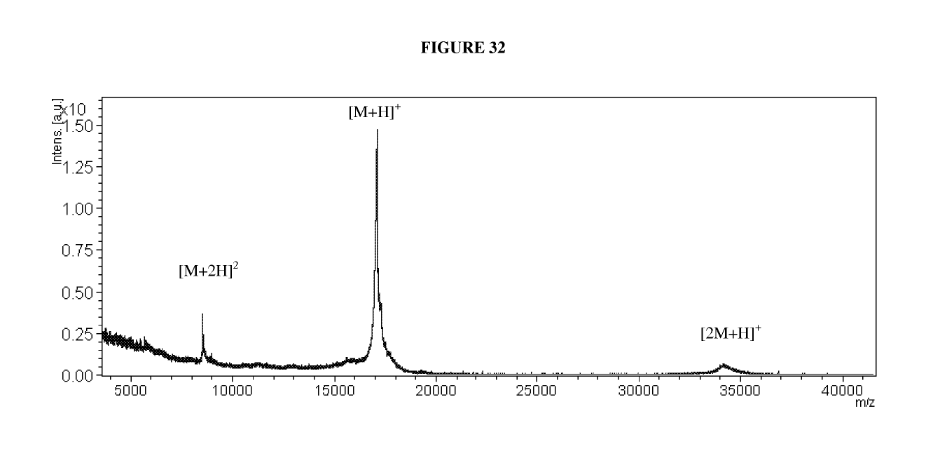

FIG. 32 shows the MALDI-TOF spectrum of the EgA1 antibody fragment.

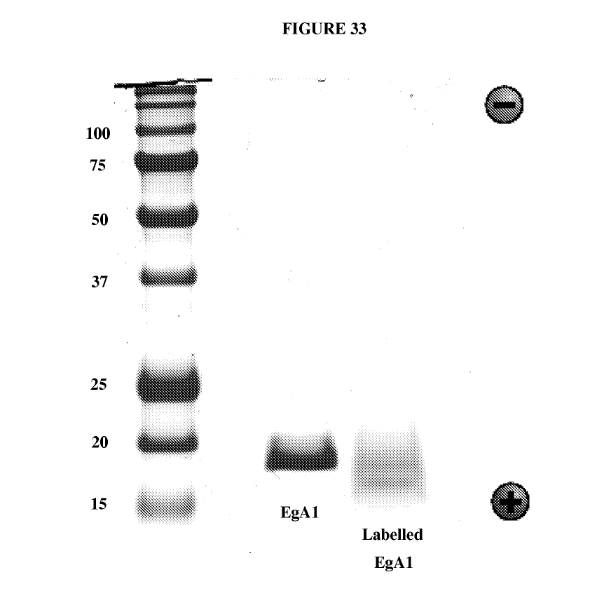

FIG. 33 shows the SDS-PAGE tricine gel of the EgA1 antibody fragment and labelled EgA1 antibody fragment.

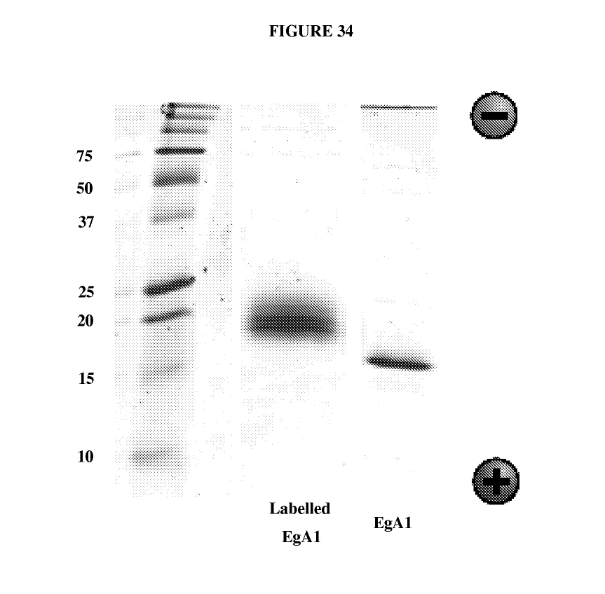

FIG. 34 shows the SDS-PAGE glycine gel of the EgA1 antibody fragment and labelled EgA1 antibody fragment.

### EXAMPLE 1

### Preparation of the Ligand L₁

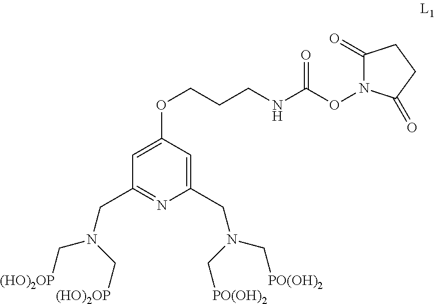

The ligand L₁ was obtained according to the following synthesis diagram:

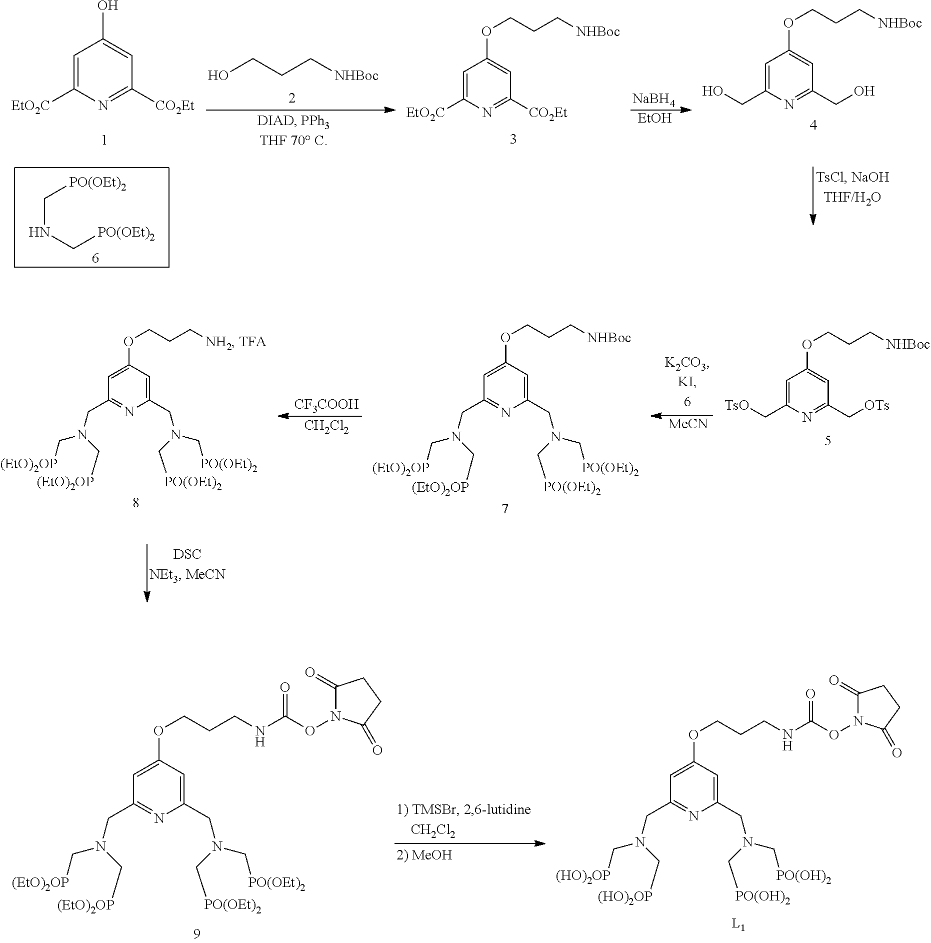

### Preparation of Compound 3:

The diester 1 and the amino alcohol 2 were prepared, respectively, according to the methods described by Célia S. Bonnet et al., in Chem. Commun. 2010, 46, 124 and by S. Machida et al. in Chem. Eur. J. 2008, 14, 1392.

Triphenylphosphine (7.20 g; 27.46 mmol) and the amino alcohol 2 (4.80 g; 27.52 mmol) dissolved in a little THF are added, under argon, to a solution of diester 1 (3.27 g; 13.70 mmol) in 325 mL of THF. DIAD (5.43 mL; 27.4 mmol) is then added dropwise. The mixture is stirred at 70° C. overnight. After evaporation of the solvent, an oil is obtained, which is purified on a silica column twice in succession, first with petroleum ether/ethyl acetate 7/3 and then with CH₂Cl₂ 100% then gradient with MeOH 0.5%, 1% then 1.5%. The weight of compound 3 obtained is 4.5 g and the yield is 83%. The characteristics of compound 3 are as follows:

R_{f}=0.63; SiO₂; CH₂Cl₂/MeOH (96/4)

¹H NMR (CDCl₃, 300 MHz): δ 1.44 (s, 9H); 1.45 (t, J=7.1 Hz, 6H); 2.05 (qt, J=6.3 Hz, 2H); 3.34 (m, 2H); 4.20 (t, J=12.6 Hz, 2H); 4.47 (q, J=7.1 Hz, 4H); 4.81 (broad s, 1H); 7.74 (s, 2H).

¹³C NMR (CDCl₃, 75 MHz): δ 14.2; 28.4; 29.4; 37.5; 62.4; 66.6; 79.4; 114.3; 150.2; 156.0; 164.7; 166.8.

IR (cm⁻¹, ATR): ν 3411, 3250, 2980, 1703, 1508, 1238, 1252, 1107, 1029, 784.

ESI⁺/MS: m/z 397.2 ([3+H]⁺, 100%).

### Preparation of Compound 4

1.01 g (26.67 mmol) of NaBH₄ is added to a solution of diester 3 (4.48 g; 11.29 mmol) in 240 mL of ethanol, then the mixture is heated under reflux. The solvent is evaporated off and then a saturated aqueous solution of NaHCO₃ is added to pH 9 and finally water is added. The aqueous phase is extracted with dichloromethane. The organic phase is dried over Na₂SO₄, filtered and concentrated. The product obtained 4 is a white solid (1.56 g; 44%) the characteristics of which are as follows:

R_{f}=0.36; SiO₂; CH₂Cl₂/MeOH (90/10)

¹H NMR (CDCl₃, 300 MHz): δ 1.44 (s, 9H); 2.01 (qt, J=6.3 Hz, 2H); 3.32 (m, 2H); 4.09 (t, J=6 Hz, 2H); 4.71 (s, 4H); 6.71 (s, 2H).

¹³C NMR (CDCl₃, 75 MHz): δ 28.5; 29.4; 37.6; 64.4; 65.7; 79.4; 105.6; 156.3; 161.2; 166.5.

IR (cm⁻¹, ATR): ν 3371, 2974, 1685, 1602, 1572, 1520, 1272, 1146, 1048, 844.

ESI⁺/MS: m/z 313.2 ([4+H]⁺, 100%); 314.2 (31%); 355.2 (33%).

### Preparation of Compound 5

At 0° C., a solution of tosyl chloride (3.55 g; 18.64 mmol) in 67 mL of THF is added dropwise to a solution of the diol 4 (1.45 g; 4.66 mmol) and of NaOH (1.12 g; 27.96 mmol) in solution in a THF/H₂O mixture (34 mL/34 mL). The reaction then develops at ambient temperature. After separating the two phases, the aqueous phase is extracted with dichloromethane. The combined organic phases are washed with a 5% aqueous solution of NaHCO₃ and then with a saturated aqueous solution of NaCl. The organic phase is then dried over Na₂SO₄, filtered and concentrated. After purification by silica column chromatography (CH₂Cl₂/MeOH: gradient 1% to 2%), the bis-tosylated compound (2.44 g) is obtained with a yield of 85% (white solid). The characteristics of compound 5 are as follows:

R_{f}=0.28; SiO₂; CH₂Cl₂/MeOH (98.5/1.5)

¹H NMR (CDCl₃, 300 MHz): δ 1.46 (s, 9H); 1.92 (m, 2H); 2.44 (s, 6H); 3.31 (m, 2H); 4.04 (t, J=6.1 Hz, 2H); 4.70 (broad s, 1H); 4.98 (s, 4H); 6.81 (s, 2H); 7.34 (d, J=8.1 Hz, 4H); 7.81 (d, J=8.3 Hz, 4H).

¹³C NMR (CDCl₃, 75 MHz): δ 21.8; 28.5; 29.4; 37.6; 66.1; 71.3; 79.6; 107.7; 128.2; 130.1; 132.8; 145.3; 155.3; 156.1; 166.7.

IR (cm⁻¹, ATR) ν: 3356, 2926, 1677, 1366, 1171, 1033, 840, 809, 669.

ESI⁺/MS: m/z 607.7 (54%); 643.2 ([5+Na]⁺, 100%); 702.3 (34%).

### Preparation of Compound 7

The amine 6 was prepared according to a procedure described by S. Aime et al., in Chem. Eur. J., 2000, 14, 6.

K₂CO₃, previously flame-treated (3.14 g; 22.72 mmol), is added to a solution of the amine 6 (2.73 g; 8.60 mmol) in 200 mL of acetonitrile, under nitrogen. After stirring for 20 min the bis-tosylated derivative 5 (2.35 g; 3.78 mmol) dissolved in acetonitrile and potassium iodide (1.29 g; 7.75 mmol) are added and the mixture is heated to 70° C. After maintaining at 70° C. overnight, the reaction has not ended. 0.92 g (2.89 mmol) of amine 6, 1.02 g (7.42 mmol) of K₂CO₃ and 0.41 g (2.47 mmol) of potassium iodide are added again and the mixture is stirred at 70° C. for 22 h. After filtration, the solvent is evaporated off; an oil is obtained, which is purified on a silica column (CH₂Cl₂/MeOH gradient: gradient 2% to 6%). The weight of the product 7 obtained is 1.75 g (50%, light yellow oil). The characteristics of compound 7 are as follows:

R_{f}=0.33; SiO₂; CH₂Cl₂/MeOH (92/8)

¹H NMR (CDCl₃, 300 MHz): δ 1.24 (t, J=7 Hz, 24H); 1.36 (s, 9H); 1.91 (m, 2H); 3.18 (m, 10H); 4.04 (m, 22H); 5.09 (broad s, 1H); 6.99 (s, 2H).

¹³C NMR (CDCl₃, 75 MHz): δ 16.4 (d, J=5.5 Hz); 28.4; 29.2; 37.5; 50.3 (dd, J=157.7 Hz, J=8.3 Hz); 61.9; 62.0 (d, J=6.2 Hz); 62.4 (t, J=8.4 Hz); 65.5; 79.0; 108.2; 156.0; 159.4; 166.3.

³¹P NMR (CDCl₃, 161.9 MHz): δ 24.60.

IR (cm⁻¹, ATR): ν 3303, 2980, 1708, 1597, 1230, 1019, 959.

ESI⁺/MS: m/z 908.1 (27%): 933.4 ([7+Na]⁺, 100%).

### Preparation of Compound 8

1.97 mL (26.6 mmol) of trifluoroacetic acid is added at 0° C., under nitrogen, to a solution of amine 7 (1.21 g; 1.33 mmol) in 15 mL of dichloromethane. The mixture then develops at ambient temperature until the starting product has disappeared. The solvent is evaporated off, and then the crude product (triflate salt) is dried under vacuum (brown oil). It is used without purification in the next step. The characteristics of compound 8 are as follows:

R_{f}=0.1; SiO₂; CH₂Cl₂/MeOH (88/12)

¹H NMR (CDCl₃, 300 MHz): δ 1.31 (t, J=7 Hz, 24H); 2.29 (m, 2H); 3.30 (m, 10H); 4.18 (m, 16H); 4.32 (broad s, 4H); 4.46 (m, 2H); 7.39 (s, 2H); 7.92 (broad s, 2H).

¹³C NMR (CDCl₃, 100 MHz): δ 16.2; 26.1; 37.1; 50.4 (dd, J=158.9 Hz, J=6.1 Hz); 57.6 (t, J=5.8 Hz); 63.5 (t, J=3.4 Hz); 67.7; 110.7; 115.5 (q, J=289.0 MHz); 155.7; 160.2 (q, J=39.7 MHz); 171.8.

³¹P NMR (CDCl₃, 161.9 MHz): δ 24.05.

IR (cm⁻¹, ATR): ν 2989, 1774, 1634, 1202, 1160, 1023, 975.

ESI⁺/MS: m/z 406.2 ([8 (free amine)+2H]²⁺, 100%); 811.3 ([8 (free amine)+H]⁺, 32%).

### Preparation of Compound 9

Triethylamine (0.33 mL; 2.39 mmol) and then DSC (612.2 mg; 2.39 mmol) dissolved in acetonitrile are added, under nitrogen, to a solution of ammonium salt 8 (1.33 mmol) in 30 mL of acetonitrile. The reaction mixture is stirred at ambient temperature until the starting product has disappeared. After evaporation of the solvent, dichloromethane is added. The organic phase is then washed several times with a saturated aqueous solution of NH₄Cl until checking by TLC indicates the disappearance of the residual DSC. The organic phase is dried over Na₂SO₄, filtered and concentrated. The product 9 obtained, which is a brown oil (821 mg; 65%), has the following characteristics:

R_{f}=0.6; SiO₂; CH₂Cl₂/MeOH (88/12)

¹H NMR (CDCl₃, 300 MHz): δ 1.30 (t, J=7.1 Hz, 24H); 2.06 (m, 2H); 2.79 (s, 4H); 3.20 (d, J=10.0 Hz, 8H); 3.39 (m, 2H); 4.21 (m, 21H); 7.52 (broad s, 2H).

¹³C NMR (CDCl₃, 75 MHz): δ 16.6 (t, J=3 Hz); 25.6; 28.4; 38.6; 50.7 (dd, J=160.2 Hz, J=7.7 Hz); 62.3 (t, J=3.7 Hz); 152.1; 169.9.

³¹P NMR (CDCl₃, 161.9 MHz): δ 24.13.

ESI⁺/MS: m/z 869.3 (77%); 952.3 ([9+H]⁺, 100%).

### Preparation of Compound L₁

1 mL (7.64 mmol) of lutidine and then 0.88 mL (7.64 mmol) of TMSBr are added to a solution of carbamate 9 (181.7 mg; 0.19 mmol) in 5 mL of dichloromethane, under nitrogen. It is stirred for about 15 hours. The solvent is then evaporated off. The crude product obtained is taken up in methanol, which is then evaporated off. This operation is carried out two more times. The solid obtained is centrifuged in the presence of dichloromethane, and then washed several times with dichloromethane and with methanol. The final product L₁ is obtained in the form of lutidinium salt (light brown solid) and has the following characteristics:

¹H NMR (D₂O, 300 MHz): δ 2.08 (m, 2H); 2.72 (s, 6H); 2.91 (s, 4H); 3.45 (m, 2H); 3.63 (d, J=12.1 Hz, 8H); 4.27 (m, 2H); 4.91 (s, 4H); 7.13 (s, 2H); 7.63 (d, J=8.1 Hz, 2H); 8.28 (t, J=8 Hz, 8H).

ESI⁺/MS (water/acetonitrile/formic acid): m/z 728.1 ([L₁+H]⁺, 100%).

### EXAMPLE 2

### Preparation of Ligand L₂

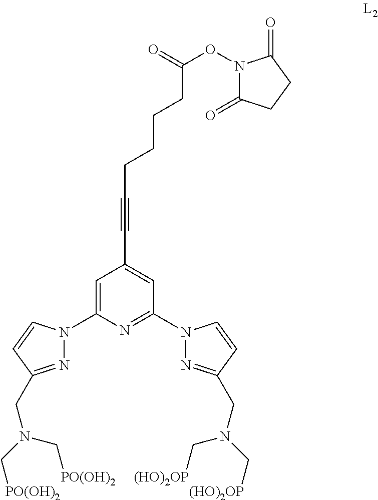

The ligand L₂ was obtained according to the following synthesis diagram:

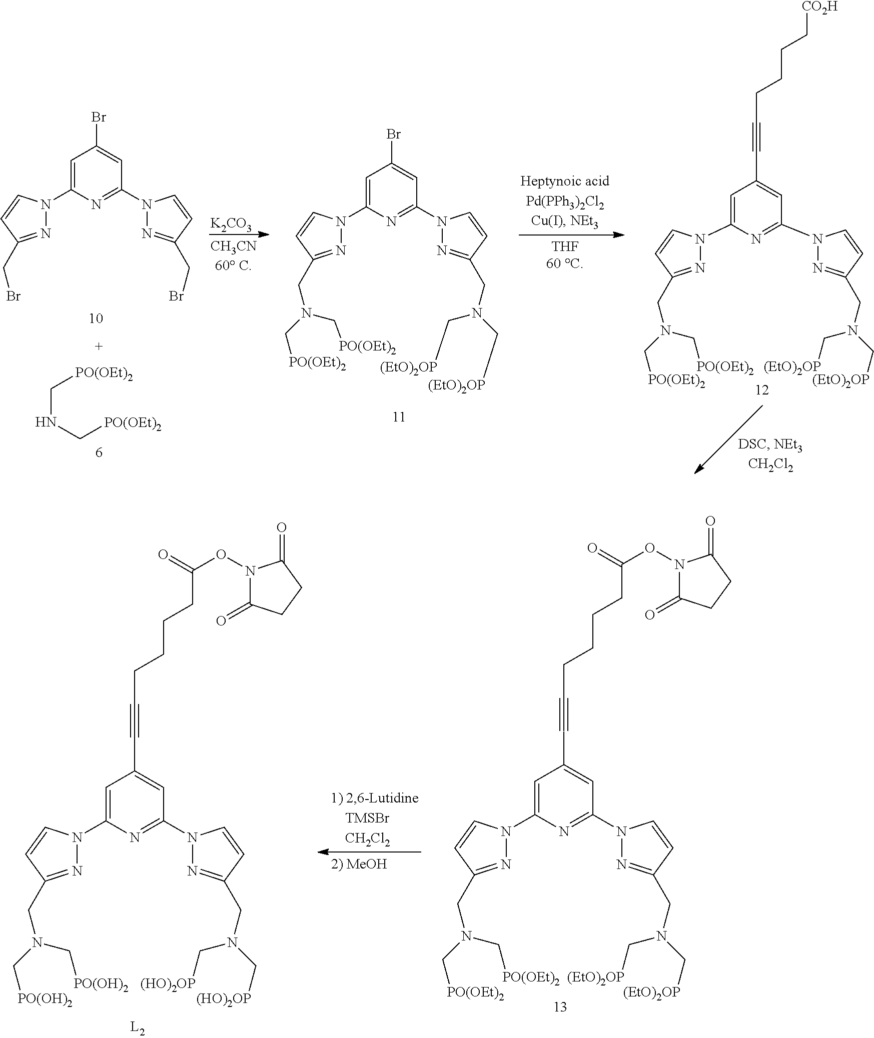

### Preparation of Compound 11:

1.5 g (4.73 mmol) of amine 6 and 1.14 g of K₂CO₃ (8.24 mmol) in 200 mL of acetonitrile dried over a solvent purifier is introduced into a two-necked flask, under a nitrogen atmosphere. The mixture is refluxed for half an hour. 0.96 g (2.06 mmol) of compound 10 (prepared according to a method described by P. Kadjane et al., in Inorg. Chem., 2009, 48, 4601) is then added. The reaction mixture is refluxed for 36 hours, until the starting reagents have been completely consumed. The solution is filtered to remove any residual K₂CO₃, and then evaporated to dryness. The oily residue was purified by silica gel column chromatography. The eluent used is a mixture of dichloromethane and methanol: CH₂Cl₂/MeOH (100/0 to 95/5). 1.2 g of compound 11 is obtained (1.26 mmol), i.e. a yield of 62%. The characteristics of compound 11 are as follows:

R_{f}=0.13; SiO₂; CH₂Cl₂/MeOH (95/5).

¹H NMR (CDCl₃, 300 MHz): δ 1.33 (t, J=70 Hz, 24H); 3.22 (d, J=10 Hz, 8H); 3.98-4.27 (m, 20H); 6.56 (d, J=2.6 Hz, 2H); 7.95 (s, 2H); 8.44 (d, J=2.6 Hz, 2H).

¹³C NMR (CDCl₃, 75 MHz): δ 16.5 (d, J=6.0 Hz); 49.0 (d, J=8.0 Hz); 51.1 (dd, J=158.0 Hz, J=8.5 Hz); 54.0; 62.0 (d, J=7.1 Hz); 109.1; 112.2; 128.0; 150.4; 153.2.

³¹P NMR (CDCl₃, 161.9 MHz): δ 23.98.

Analyses calculated for C₃₃H₅₈BrN₇O₁₂P₄.H₂O: C, 41.00; H, 6.26; N, 10.14. Found: C, 40.83; H, 6.44; N, 9.95.

IR (cm⁻¹, ATR): ν 3486, 2982, 2928, 2907, 1679, 1589, 1463-1388, 1211, 1017.

EI⁺/MS: m/z 949.3 ([11+H]⁺, 100%).

### Preparation of Compound 12:

1.12 g (1.20 mmol) of compound 11, 182 mg (1.44 mmol) of 6-heptynoic acid at 90% and 42 (0.06 mmol) mg of [Pd(PPh₃)₂Cl₂], all in solution in 80 mL of freshly distilled THF and 24 mL of triethylamine are introduced into a 250-mL two-necked flask. The solution is degassed by passing through a continuous stream of nitrogen for 30 min 22.9 mg (0.1 mmol) of CuI is added and then the reaction mixture is heated at 60° C. for 15 hours. The solvent is removed by evaporation and successive co-evaporations with dichloromethane. The oily residue is taken up in 250 mL of a CH₂Cl₂/H₂O mixture (50/50), and then the aqueous phase is extracted. The organic phase is washed with saturated NaCl solution, and then dried over Na₂SO₄. The residue is purified by silica gel column chromatography. The eluent used is a CH₂Cl₂/MeOH mixture (100/0 to 93/7). 1.01 g (1.02 mmol) of compound 12 is obtained in the form of a brown oil, i.e. a yield of 85%. Compound 12 has the following characteristics:

R_{f}=0.27; SiO₂; CH₂Cl₂/MeOH (90/10).

¹H NMR (CDCl₃, 300 MHz): δ 1.33 (t, J=7.0 Hz, 24H); 1.70 (m, 2H); 1.95 (m, 2H); 2.43 (m, 2H); 2.51 (m, 2H); 3.26 (d, J=10 Hz, 8H); 4.05-4.35 (m, 20H); 6.50 (d, J=2.6 Hz, 2H); 7.82 (s, 2H); 8.45 (d, J=2.6 Hz, 2H).

¹³C NMR (CDCl₃, 75 MHz): δ 16.5 (d, J=6.1 Hz); 19.1; 19.4; 24.0; 27.2; 50.1 (dd, J=159.9 Hz, J=9.4 Hz); 58.3; 62.2 (d, J=7.0 Hz); 96.6; 108.8; 111.4; 127.67; 137.4; 150.0; 152.1; 175.4; 206.9.

³¹P NMR (CDCl₃, 161.9 MHz): δ 24.73.

Analyses calculated for C₄₀H₆₇N₇O₁₄P₄: C, 48.34; H, 6.79; N, 9.86. Found: C, 48.52; H, 7.12; N, 9.65.

IR (cm⁻¹, ATR): ν 2233, 1720, 1211, 1019, 987, 795.

EI⁺/MS: m/z 994.4 ([12+H]⁺, 100%).

### Preparation of Compound 13:

300 mg (0.30 mmol) of compound 12, 175 mg (0.68 mmol) of N,N′-disuccinimidyl carbonate, 240 μL of triethylamine and 45 mL of CH₂Cl₂ are introduced into a 100-mL single-necked flask. The reaction mixture is stirred under nitrogen atmosphere for 12 hours. The reaction mixture is then evaporated to dryness, taken up in 100 mL of CH₂Cl₂, and washed successively with a saturated aqueous solution of NH₄Cl and with water. The organic phase is dried over Na₂SO₄, filtered, and then evaporated to dryness. 320 mg of compound 13 is obtained (0.29 mmol; 97%) in the form of a pale yellow oil the characteristics of which are as follows:

¹H NMR (CDCl₃, 300 MHz): δ 1.26 (t, J=7.1 Hz, 24H); 1.70-1.83 (m, 2H); 1.86-1.98 (m, 2H); 2.52 (t, J=6.9 Hz, 2H); 2.68 (t, J=7.2 Hz, 2H); 2.83 (s, 47H); 3.21 (d, J=10.1 Hz, 8H); 4.03-4.24 (m, 20H); 6.52 (d, J=2.7 Hz, 2H); 7.73 (s, 2H); 8.44 (d, J=2.7 Hz, 2H).

¹³C NMR (CDCl₃, 75 MHz): δ 16.6 (d, J=6.1 Hz); 19.2; 23.9; 25.7; 27.4; 30.6; 50.1 (dd, J=158.4 Hz, J=9.7 Hz); 62.1 (d, J=7.2 Hz); 79.0; 95.9; 108.8; 111.3; 127.9; 137.2; 150.2; 152.7; 168.4; 169.2.

³¹P NMR (CDCl₃, 161.9 MHz): δ 24.49.

IR (cm⁻¹, ATR): ν 2234, 1841, 1784, 1740, 1715, 1209, 962, 742.

### Preparation of Ligand L₂:

224 mg (2.09 mmol) of lutidine and 320 mg (2.09 mmol) of TMSBr are introduced into a 50-mL single-necked flask containing 57 mg of compound 13 (0.053 mmol) in solution in 10 mL of dichloromethane. The reaction mixture is stirred at ambient temperature for 12 hours, and then the volatile substances are evaporated off at ambient temperature under reduced pressure. The TMSBr is co-evaporated with dichloromethane (the operation is repeated twice). A white precipitate is then obtained. 10 mL of methanol is added, evaporating immediately, still at ambient temperature, under reduced pressure. The operation is repeated twice. The whitish precipitate obtained is washed with dichloromethane, and then methanol to give the ligand L₂ as a brown powder, in the form of lutidinium monophosphonate and the characteristics of which are as follows:

¹H NMR (D₂O—NaOD, 200 MHz): δ 1.28 (m, 4H); 1.83 (m, 2H); 2.15 (m, 2H); 2.30 (d, J=12.1 Hz, 8H); 2.91 (s, 6H); 3.66 (s, 4H); 6.37 (s, 2H); 6.70 (d, J=7.6 Hz, 2H); 7.22 (m, 3H); 8.18 (s, 2H).

³¹P NMR (CDCl₃, 161.9 MHz): δ 16.89.

Analyses calculated for C₂₈H₃₈N₈O₁₆P₄.C₇H₉N.4H₂O: C, 40.20; H, 5.30; N, 12.05. Found: C, 40.26; H, 5.24; N, 12.07.

IR (cm⁻¹, ATR): ν 2157, 1837, 1781, 1736, 1611, 1203, 987, 795.

ESI⁻/MS: m/z 865.15 ([L₂−H]⁻, 30%); 768.14 ([L₂−NHS]⁻, 100%).

### EXAMPLE 3

### Preparation of Ligand L₃

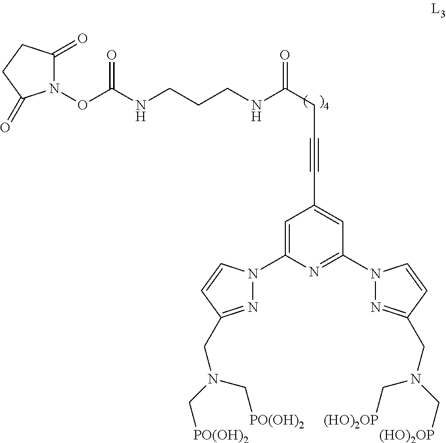

The ligand L₃ was obtained according to the following synthesis diagram:

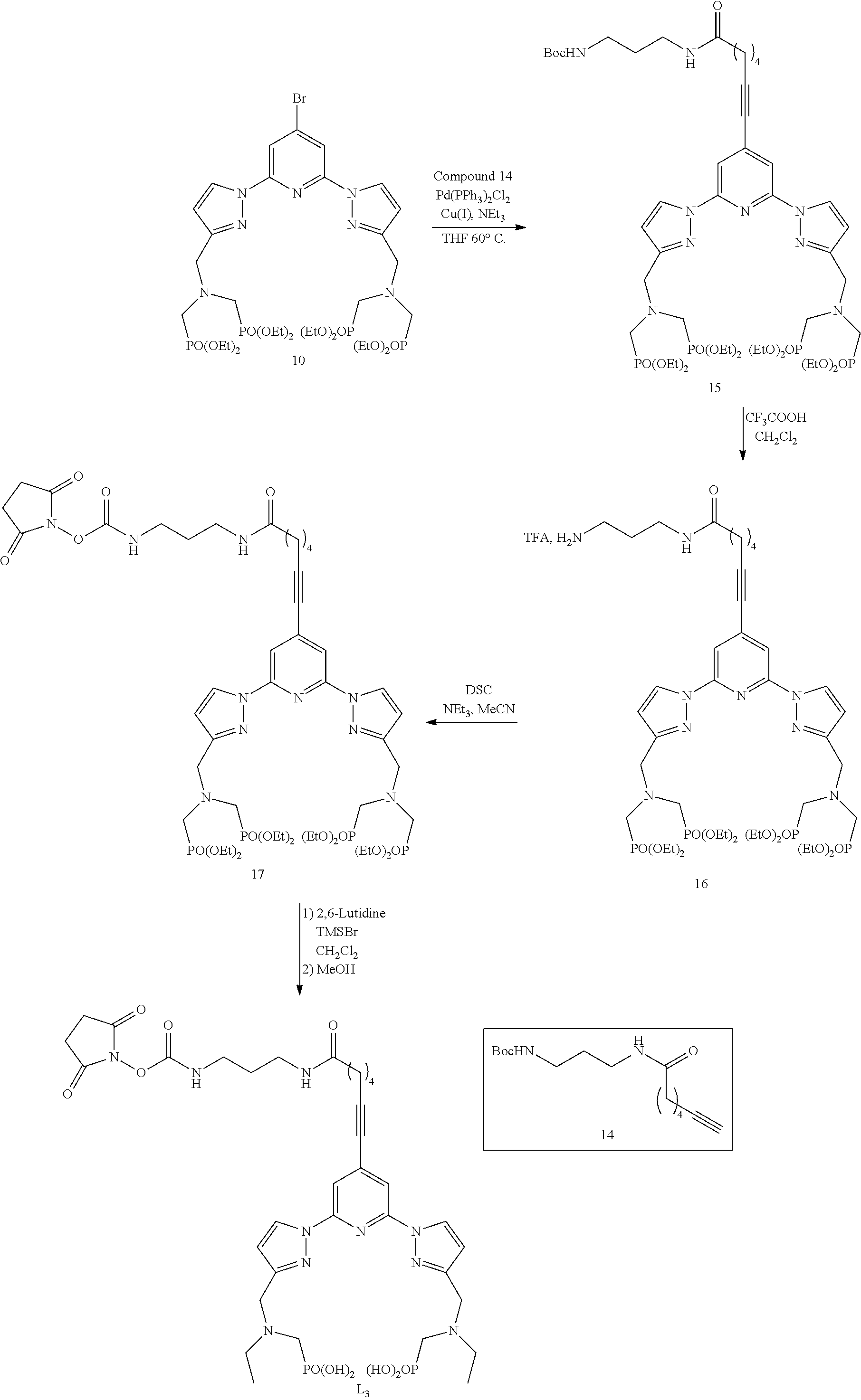

### Preparation of Compound 15

400 mg (0.42 mmol) of compound 10, 191 mg (0.67 mmol) of compound 14 and 15 mg (0.021 mmol) of [Pd(PPh₃)₂Cl₂], all in solution in 20 mL of freshly distilled THF and 8 mL of triethylamine are introduced into a 250-mL two-necked flask. The solution is degassed by passing through a continuous stream of nitrogen for 30 min 8 mg (0.04 mmol) of CuI is added and then the reaction mixture is heated at 60° C. for 15 hours. The solvents are removed by evaporation and then co-evaporations with dichloromethane. The oily residue obtained is taken up in 60 mL of a CH₂Cl₂/H₂O mixture (50/50), and then the aqueous phase is extracted. The organic phase is washed with saturated NaCl solution, and then dried over Na₂SO₄. The residue is purified by silica gel column chromatography. The eluent used is CH₂Cl₂/MeOH (100/0 to 93/7). 428 mg (0.37 mmol) of compound 15 is obtained in the form of a brown oil, i.e. a yield of 88%. Compound 15 has the following characteristics:

R_{f}=0.17; SiO₂; CH₂Cl₂/MeOH (90/10).

¹H NMR (CDCl₃, 300 MHz): δ 1.31 (t, 24H, J=7.1 Hz); 1.41 (s, 9H); 1.54-1.74 (m, 4H); 1.75-1.92 (m, 2H); 2.25 (t, J=7.2 Hz, 2H); 2.47 (t, J=6.8 Hz, 2H); 3.34-3.06 (m, 12H); 3.93-4.28 (m, 22H); 6.52 (d, J=2.6 Hz, 2H); 7.72 (s, 2H); 8.44 (d, 2H, J=2.6 Hz).

¹³C NMR (CDCl₃, 75 MHz): δ 16.4 (d, J=6.1 Hz); 19.3; 25.0; 27.9; 28.4; 30.2; 35.8; 36.1; 37.0; 49.9 (d, J=159.4 Hz); 53.9; 62.0; 62.1; 78.6; 79.2; 96.6; 108.6; 111.2; 127.8; 137.3; 150.0; 152.5; 172.9.

³¹P NMR (CDCl₃, 161.9 MHz): δ 24.50.

Analyses calculated for C₄₈H₈₃N₉O₁₅P₄.2MeOH: C, 49.46; H, 7.55; N, 10.38. Found: C, 49.31; H, 7.39; N, 10.01.

ESI⁺/MS: m/z 1172.5 ([15+Na]⁺, 100%).

### Preparation of Compound 16:

120 mg of compound 15 (0.10 mmol) in solution in 10 mL of CH₂Cl₂ is introduced into a 100-mL single-necked flask. 78 μL (1 mmol) of trifluoroacetic acid is added and then the reaction mixture is stirred at ambient temperature for 15 hours. The volatile compounds are removed by evaporation and then successive co-evaporations with dichloromethane. 150 mg of compound 16 (ammonium triflate) is obtained, in the form of a brown oil the characteristics of which are as follows:

¹H NMR (CD₃OD, 300 MHz): δ 1.20-1.26 (m, 24H); 1.52-1.65 (m, 2H); 1.66-1.81 (m, 2H); 1.85-2.01 (m, 2H); 2.13-2.27 (m, 2H); 2.39-2.54 (m, 2H); 2.79-3.01 (m, 2H) 3.07-3.28 (m, 18H); 3.93-4.19 (m, 16H); 6.51 (m, 2H); 7.63 (s, 2H); 8.55-8.66 (m, 2H).

³¹P NMR (CDCl₃, 161.9 MHz): δ 24.83.

IR (cm⁻¹, ATR): ν 2158, 1676, 1200, 1015, 797.

ESI⁺/MS: m/z 1050.5 ([16+H]⁺, 100%).

### Preparation of Compound 17:

57 mg (0.05 mmol) of compound 16, 27 mg (0.09 mmol) of N,N′-disuccinimidyl carbonate, 50 μL of triethylamine and 15 mL of CH₂Cl₂ are introduced into a 50-mL single-necked flask. The reaction mixture is stirred under nitrogen atmosphere for 12 hours. The reaction mixture is then evaporated to dryness, taken up in 20 mL of CH₂Cl₂, and washed successively with a saturated aqueous solution of NH₄Cl and with water. The organic phase is dried over Na₂SO₄, filtered, and then evaporated to dryness. 50 mg (0.04 mmol) of compound 17 is obtained in the form of a pale yellow oil, the characteristics of which are as follows:

¹H NMR (CDCl₃, 300 MHz): 1.08-1.48 (m, 26H); 1.71 (m, 2H); 1.83 (m, 2H); 2.28 (t, J=7.3 Hz, 2H); 2.50 (t, J=6.7 Hz, 2H); 2.79 (s, 4H); 3.10-3.44 (m, 12H); 4.03-4.26 (m, 20H); 6.52 (d, J=2.5 Hz, 2H); 7.73 (s, 2H); 8.45 (d, J=2.6 Hz, 2H).

¹³C NMR (CDCl₃, 75 MHz): δ 16.7 (d, J=6.2 Hz); 19.5; 25.3; 25.7; 28.1; 29.8; 36.3; 38.9; 50.2 (dd, J=159 Hz, J=8.4 Hz); 53.9; 54.1; 62.3 (d, J=6.9 Hz); 108.9; 111.4; 128.0; 150.0; 152.7; 170.0; 173.8; 180.2; 186.1; 195.1; 205.2.

³¹P NMR (CDCl₃, 161.9 MHz): 324.54.

IR (cm⁻¹, ATR): ν 2239, 1812, 1781, 1736, 1611, 1204, 1060, 794.

ESI⁺/MS: m/z 1213.4 ([17+Na]⁺, 90%).

### Preparation of Ligand L₃:

204 mg (2.21 mmol) of lutidine and 232 mg (1.68 mmol) of TMSBr are added to a 50-mL single-necked flask containing 50 mg of compound 17 (42 μmol) in solution in 10 mL of dichloromethane. The reaction mixture is stirred at ambient temperature for 12 hours, and then the volatile substances are evaporated off at ambient temperature under reduced pressure. The TMSBr is co-evaporated with dichloromethane (the operation is carried out twice). A white precipitate is then obtained. 10 mL of methanol is added, evaporating immediately, still at ambient temperature, under reduced pressure. The ligand L₃, obtained in the form of lutidinium monophosphonate, has the following characteristics:

¹H NMR (D₂O—NaOD, 300 MHz): δ 1.47-1.89 (m, 6H); 2.30 (m, 2H); 2.41 (m, 4H); 2.54 (m, 2H); 2.70 (d, J=11.2 Hz, 8H); 3.01 (m, 2H); 3.20 (m, 2H); 4.08 (s, 4H); 6.78 (s, 2H); 7.07 (d, J=7.8 Hz, 1H); 7.52-7.69 (m, 2.5H); 8.58 (s, 2H).

³¹P NMR (CDCl₃, 161.9 MHz): δ 16.68.

ESI⁻/MS: m/z: 965.2 ([L₃−H]⁻, 30%).

### EXAMPLE 4

### Preparation of Ligand L₄

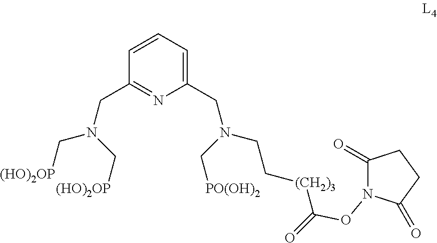

The ligand L₄ was obtained according to the following synthesis diagram:

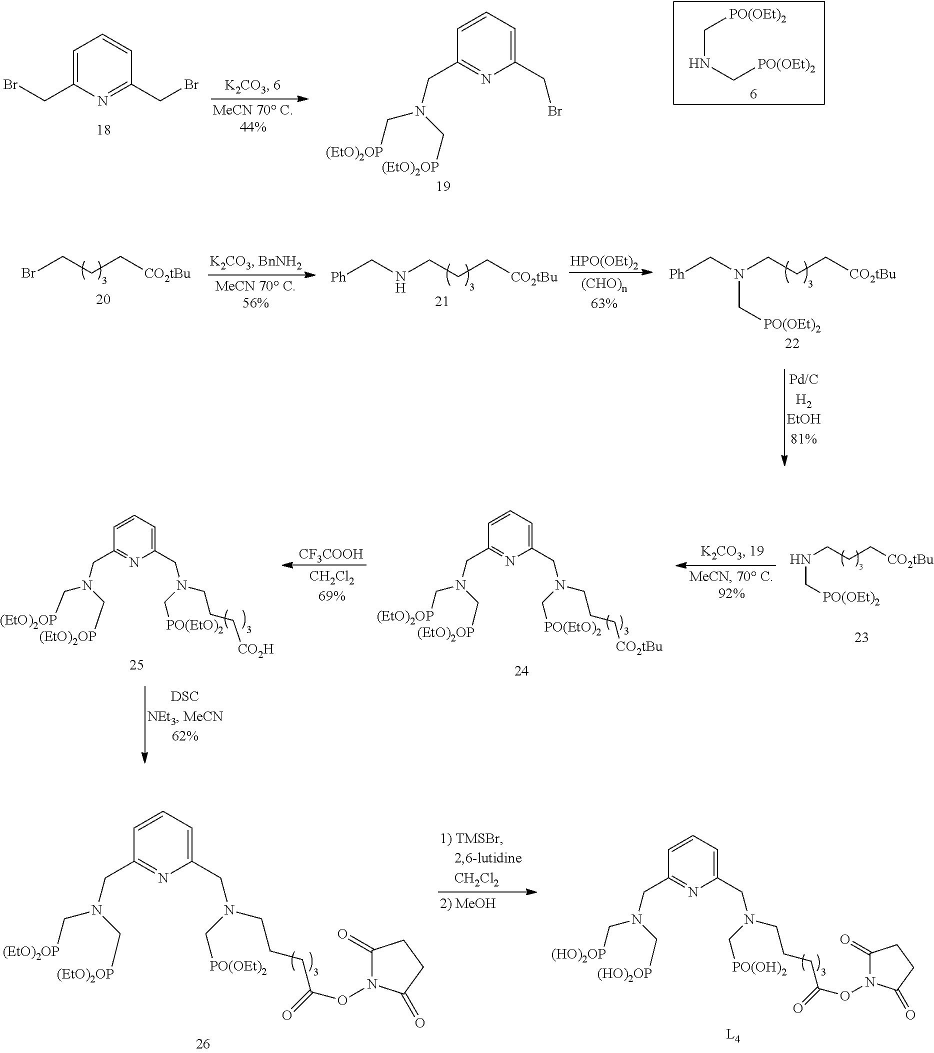

### Preparation of Compound 19

2,6-bis(bromomethyl)pyridine 18 was prepared according to a procedure described by T. Vermoden et al., in Tetrahedron, 2003, 59, 5039.

The amine 6 (359.13 mg; 1.13 mmol) is added to a solution of K₂CO₃, previously flame-treated (391.13 mg; 2.83 mmol) in 20 mL of acetonitrile, under nitrogen, and then 2,6-bis(bromomethyl)pyridine 18 (300 mg; 1.13 mmol) is added. The mixture is heated at 65° C. for a further 6 h, and then left to stand overnight at ambient temperature. After filtration, the solvent is evaporated off; the crude product is purified on a silica column (CH₂Cl₂/MeOH gradient from 100/0 to 94/6). The weight of the product 19 obtained is 253 mg (44%, oil). The characteristics of compound 19 are as follows:

R_{f}=0.6; SiO₂; CH₂Cl₂/MeOH (90/10)

¹H NMR (CDCl₃, 300 MHz): δ 1.29 (t, J=7.3 Hz, 12H); 3.23 (d, J=9.7 Hz, 4H); 4.11 (m, 10H); 4.51 (s, 2H); 7.31 (d, J=7.6 Hz, 1H); 7.51 (d, J=7.6 Hz, 1H); 7.68 (t, J=7.8 Hz, 1H).

¹³C NMR (CDCl₃, 75 MHz): δ 16.6 (t, J=3.1 Hz); 34.0; 50.4 (dd, J=159.8 Hz, J=7.4 Hz); 62.2; 122.1; 123.1; 137.6; 156.0; 158.8.

³¹P NMR (CDCl₃, 161.9 MHz): δ 24.4.

IR (cm⁻¹, ATR): ν 3415, 962-1016, 1592-1675, 1392.

ESI⁺/MS (CH₂Cl₂): m/z 363.0 (22%); 417.9 (44%); 501.1 ([19+H]⁺, 54%); 503.1 ([19+H]⁺, 54%); 523.1 ([19+Na]⁺, 70%); 525.1 ([19+Na]⁺, 70%); 598.2 (17%); 737.0 (42%); 752.3 (100%); 759.3 (94%).

### Preparation of Compound 21

1.20 g (4.77 mmol) of tert-butyl 6-bromohexanoate 20 (prepared according to a procedure described by M. S. Shchepinov in European Patent Application EP1506959A2, 2005) and 1.02 g of benzylamine (9.52 mmol) are added to a solution of K₂CO₃, previously flame-treated (1.98 g; 14.31 mmol) in 20 mL of acetonitrile, under nitrogen, and then the mixture is maintained at 65° C. for 24 h. After filtration, water is added and then the aqueous phase is extracted with dichloromethane. The organic phase is dried over Na₂SO₄, filtered and concentrated. The crude product is purified on a silica column (CH₂Cl₂/MeOH gradient from 100/0 to 90/10). The weight of the product 21 obtained is 1.32 g (56%, oil).

¹H NMR (CDCl₃, 300 MHz): δ 1.44 (m, 15H); 2.16 (t, J=7.3 Hz, 2H); 2.58 (t, J=7.2 Hz, 2H); 3.73 (s, 2H); 7.19-7.27 (m, 5H).

¹³C NMR (CDCl₃, 75 MHz): δ 25.1; 26.9; 28.2; 29.9; 35.6; 49.3; 54.1; 80.1; 127.0; 128.3; 128.5; 140.5; 173.3.

Analyses calculated for C₁₇H₂₇NO₂: C, 73.61; H, 9.81; N, 5.05. Found: C, 73.59; H, 9.90; N, 5.07.

IR (cm⁻¹, ATR): ν 2931; 1604; 1727.

ESI⁺/MS (CH₂Cl₂): m/z 222.1 ([21−C₄H₉+H]⁺, 66%); 278.2 ([21+H]⁺, 100%); 392.3 (16%); 432.2 (31%); 481.3 (31%); 709.5 (19%).

### Preparation of Compound 22

300 mg (1.08 mmol) of compound 21 and 223.21 mg of diethyl phosphite (1.62 mmol) are mixed and then formaldehyde (37% in water) (174.63 mg; 2.15 mmol) is added dropwise. The reaction mixture is heated at 100° C. for 3 h. After evaporation to dryness, the crude product is purified on a silica column (CH₂Cl₂/MeOH gradient from 100/0 to 99/1). 290 mg of the amine 22 is obtained in the form of an oil, i.e. a yield of 63%. The product 22 has the following characteristics:

¹H NMR (CDCl₃, 300 MHz): δ 1.21-1.57 (m, 21H); 2.17 (t, J=7.5 Hz, 2H); 2.60 (t, J=7.1 Hz, 2H); 2.86 (d, J=10.3 Hz, 2H); 3.74 (s, 2H); 4.09 (qt, J=9.6 Hz, 4H); 7.17-7.33 (m, 5H).

¹³C NMR (CDCl₃, 75 MHz): δ 16.6 (d, J=5.8 Hz); 25.1; 26.7; 28.2; 32.8; 35.7; 49.2 (d, J=156.2 Hz); 54.9 (d, J=8.3 Hz); 59.7 (d, J=8.3 Hz); 61.8 (d, J=6.8 Hz); 80.0; 127.1; 128.3; 129.1; 139.1; 173.2.

³¹P NMR (CDCl₃, 161.9 MHz): δ 25.8.

IR (cm⁻¹, ATR): ν 2932; 1727; 1679; 1052.

### Preparation of Compound 23

Using a hydrogen generator (0.1 bar), hydrogen is bubbled into a solution containing 250 mg (0.584 mmol) of the amine 22, and 150 mg of palladium on charcoal in 20 mL of ethanol. The mixture is heated under reflux for 3.5 h. After filtration on Celite, and evaporation of the solvent, 160 mg of compound 23 is obtained in the form of an oil (i.e. a yield of 81%). The product 23 has the following characteristics:

¹H NMR (CDCl₃, 300 MHz) 1.31 (m, 8H); 1.41-1.61 (m, 13H); 2.18 (t, J=7.5 Hz, 2H); 2.65 (t, J=7.1 Hz, 2H); 2.95 (d, J=12.5 Hz, 2H); 4.13 (m, 4H).

¹³C NMR (CDCl₃, 75 MHz): δ 16.6 (d, J=5.4 Hz); 25.0; 26.7; 28.2; 29.5; 35.6; 45.3 (d, J=154.3 Hz); 51.3 (d, J=15.3 Hz); 62.1 (d, J=6.7 Hz); 80.1; 173.2.

³¹P NMR (CDCl₃, 161.9 MHz): δ 26.4.

ESI⁺/MS (CH₂Cl₂): m/z 338.2 ([23+H]⁺, 100%); 369.0 (45%); 373.6 (56%); 675.5 (21%); 780.4 (96%).

### Preparation of Compound 24

Compound 23 (125 mg; 0.37 mmol) and the mono-substituted product 19 (250 mg; 0.498 mmol) are added to a solution of K₂CO₃, previously flame-treated (2.17 g; 15.70 mmol) in 8 mL of acetonitrile, under nitrogen, and then the mixture is heated at 70° C. overnight. After filtration, the solvent is evaporated off; the crude product is purified on a silica column (CH₂Cl₂/MeOH gradient from 100/0 to 95/5). The weight of the product 24 obtained is 259 mg (92%).

¹H NMR (CDCl₃, 200 MHz) 1.26-1.59 (m, 37H); 2.18 (m, 2H); 2.63 (m, 2H); 2.94 (d, J=10.4 Hz, 2H); 3.21 (d, J=9.9 Hz, 2H); 3.88 (distorted s, 2H); 4.11 (m, 16H); 7.44 (m, 2H); 7.64 (m, 1H, H₄).

³¹P NMR (CDCl₃, 161.9 MHz): δ 26.1.

IR (cm⁻¹, ATR): ν 3476-2933, 1727, 1020.

ESI⁺/MS (CH₂Cl₂): m/z 780.4 ([24+Na]⁺, 100%).

### Preparation of Compound 25

0.78 mL (10.54 mmol) of trifluoroacetic acid is added at 0° C., under argon, to a solution of ester 24 (400 mg; 0.527 mmol) in dichloromethane. The mixture then develops at ambient temperature overnight. The solvent is evaporated off, and the crude product is purified on a silica column (CH₂Cl₂/MeOH gradient from 100/0 to 95/5). The weight of acid 25 obtained is 254 mg (69%). The characteristics of compound 25 are as follows:

¹H NMR (Acetone-d⁶, 300 MHz): δ 1.26-1.33 (m, 20H); 1.49-1.66 (m, 4H); 2.05 (m, 2H); 2.24 (t, J=7.2 Hz, 2H); 2.80 (m, 2H); 3.31 (m, 4H); 4.08-4.27 (m, 16H); 7.56 (m, 2H); 7.93 (t, J=7.8 Hz, 1H).

³¹P NMR (CDCl₃, 161.9 MHz): δ 23.7-26.0 (broad m).

IR (cm⁻¹, ATR): ν 3477-2934, 1727, 1020.

ESI⁺/MS (CH₂Cl₂): m/z 430.2 (6%); 552.3 (8%); 594.6 (8%); 702.3 ([25+H]⁺, 100%); 757.2 (27%); 774.4 (14%); 934.15 (10%).

### Preparation of Compound 26

152.42 mg (0.595 mmol) of N,N′-disuccinimidyl carbonate and 0.19 mL (1.35 mmol) of triethylamine are added to 190 mg (0.270 mmol) of acid 25 in 10 mL of CH₂Cl₂. At the end of the reaction, the reaction mixture is evaporated to dryness, taken up in CH₂Cl₂, and then washed several times with a saturated aqueous solution of NH₄Cl until the DSC disappears (checking by TLC). The organic phase is dried over Na₂SO₄, filtered, and then evaporated to dryness. 133.5 mg of the crude N-hydroxysuccinimide ester 26 is obtained (62%), and the characteristics of which are as follows:

¹H NMR (CDCl₃, 200 MHz): δ 1.26-1.75 (m, 23H); 2.59 (m, 4H); 2.83 (distorted s, 5H); 2.94 (d, J=10.6 Hz, 2H); 3.21 (d, J=10.2 Hz, 2H); 3.88 (distorted s, 2H); 4.11 (m, 16H); 7.43 (m, 2H); 7.65 (m, 1H).

³¹P NMR (CDCl₃, 161.9 MHz): δ 24.5.

ESI⁺/MS (CH₂Cl₂): m/z 799.3 ([26+H]⁺, 100%); 822.0 (20%).

### Preparation of Compound L₄

0.16 mL (1.40 mmol) of lutidine and then 0.15 mL (1.12 mmol) of TMSBr are added to a solution of ester 26 (30 mg; 0.0375 mmol) in dichloromethane, under argon. It is stiffed for about 22 hours. The solvent is then evaporated. The crude product obtained is taken up in methanol, which is then evaporated off. This operation is carried out two more times. The solid obtained is washed several times with dichloromethane and with methanol. The final product L₄ is obtained in the form of lutidinium salt (brown solid). The characteristics of the crude compound L₄ are as follows:

¹H NMR (D₂O, 300 MHz): δ 1.38 (m, 3H); 1.62 (m, 3H); 1.84 (m, 3H); 2.39 (m, 2H); 2.70 (s, 3H); 2.77 (s, 4H); 3.28-3.75 (m, 13H); 4.92 (m, 3H); 7.45-7.64 (m, 4H); 7.99 (t, J=7.8 Hz, 1H); 8.25 (m, 0.4H).

### EXAMPLE 5

### Labelling of Streptavidin with the Ligand L₂

### 5.1. Procedure

Streptavidin (0.2 mg; 3.3 nmol) in solution in 20 μL of water is added to a 1-mL flask containing ligand L₂ (0.13 mg; 13.2×10⁻⁹ mol) in 200 μL of an aqueous solution of ammonium bicarbonate buffer (pH=8.05; C=200·10⁻³ mol·L⁻¹). The solution obtained is stirred at ambient temperature for 15 h. The reaction mixture is concentrated and the excess ligand is removed by ultracentrifugation (VIVASPIN 500 Sartorius Stedim, cut-off 5000MWCO PES, speed of rotation: 1500 r.p.m.). The solution is subjected to 5 cycles of centrifugation, each of two minutes, with addition of 100 μL of the buffer. After these operations, 100 μL of centrifugate is collected. The latter is analysed by two methods: UV-Visible absorption spectroscopy and mass spectrometry.

### 5.2. Analysis by Mass Spectroscopy:

The centrifugate was analysed by MALDI-MS mass spectrometry.

Matrix: α-cyano-4-hydroxycinnamic acid.
Method of deposition: Dried droplet.

The mass spectrum of unlabelled streptavidin (FIG. 1) shows a main peak at 13038 units of m/z. Analysis of the mass spectrum of labelled streptavidin (FIG. 2) shows 3 main peaks at 13049, 13860 and 14549, corresponding respectively to a monomer of unlabelled streptavidin (Strep), a monomer labelled with a ligand L₂ (Strep-(L₂)) and a monomer labelled twice (Strep(L₂)₂).

### 5.3. UV-Visible Spectroscopy:

Titration of 25 μL of the centrifugate, or 0.83 nmol of StrepL₂, is carried out with a solution of TbCl₃.6H₂O (C=5.36×10⁻⁵ mol·L⁻¹).

Complexing of the metal by the phosphonate groups of the ligands bound to the streptavidin shifts the absorption maximum of the sample from 320 nm to 329 nm. This wavelength is reached for 210 μL of Tb added, or 11.3 nmol of StrepTbL₂ for forming 1:1 complexes. From this an average degree of labelling of 13 ligands L₂ per streptavidin id deduced.

### EXAMPLE 6

### Labelling of the B28.13 Antibody with the Ligand L₁

The B28.13 antibody is an antibody directed against tenascin, an extracellular glycoprotein that develops around tumours (such as colon cancer).

### 6.1 HPLC Analysis

A method was developed for analysing the antibody by HPLC. The analyses were carried out on an Alliance 2695 HPLC chain (Waters) equipped with a UV detector with a Waters 2996 diode array. The separations were carried out on a Poros R1 1×150 mm column (10 μm) (Applied Biosystems). The flow was fixed at 0.4 mL/min, column temperature was maintained at 50° C. and the gradient used was: solvent A: acidified water (0.1% TFA), solvent B: acidified acetonitrile (0.1% TFA). The gradient begins at 5% of solvent B, is maintained for 5 minutes and is then increased linearly to 85% over 25 minutes, then increased again to 95% over one minute and maintained at 95% for 3 minutes. A step of column equilibration in 5% of solvent B follows elution. Elution is monitored at 210 nm (FIG. 3).

### 6.2 MALDI-TOF MS Analyses

MALDI-TOF analyses were carried out on the antibody. The solution of antibody is desalted on ZipTip C18 (Millipore). A quantity of 70 pmol of antibody is loaded and then eluted in 5 μL H₂O/acetonitrile/HCOOH 20/75/5 (v/v/v). 0.6 μL of the sample (about 8 pmol) are deposited in triplicate on a MALDI MTP 348 plate (Brucker Daltonics) with 0.6 μL of sinapinic acid (at 2 mg/mL in 50% of acetonitrile). The analyses were carried out in positive mode and the mass spectrometer (Autoflex, Brucker Daltonics) was calibrated over an m/z range from 20000 to 190000 with BSA. The result is shown in FIG. 4.

This result allowed an estimate of the mass of the B28.13 antibody around 148000 Da.

### 6.3 Labelling of the Antibody

The labelling experiment was carried out on 230 μg (i.e. 1.55×10⁻⁹ mol) of the B28.13 antibody, to which 0.86 mg of the solid ligand L₁ (purity 30%) i.e. 3.5×10⁻⁷ mol was added. This means 230 equivalents of ligand L₁ for each antibody. The pH was adjusted to 7 with PBS buffer and the reaction mixture was stirred for one hour at ambient temperature.

The reaction mixture was then purified in order to remove the excess ligand L₁ by ultrafiltration on vivaspin500 modules (Sartorius) with a cut-off of 30 kDa and washed several times (20 cycles) with a Tris-HCl 0.01 M buffer at pH 7.

The reaction mixture was then deposited, after desalting on ZipTip C18, on a MALDI plate as described above. The result is shown in FIG. 5.

The mass spectrum shows a slight shift of the [M+H]⁺ peak to the right due to the mass of the ligand L₁, which is added to that of the antibody. At the level of the maximum, there is a difference of 800 Da, which would correspond to 1 ligand L₁ per antibody.

6.4 Activity of the Antibody after Labelling

The activity of the labelled B28.13 antibody was tested following labelling on a section from human colon cancer, then revealed by a secondary antibody containing a dye. This test revealed that the antibody still had affinity for its target (FIG. 6).

### EXAMPLE 7

### Labelling of the dreg55 Antibody with the Ligand L₂

The dreg55 antibody is a monoclonal antibody possessing very strong affinity for L-selectin.

### 7.1 HPLC Analysis

A method was developed for analysing the antibody by HPLC. The analyses were carried out on an Alliance 2695 HPLC chain (Waters) equipped with a UV detector with a Waters 2996 diode array. The separations were carried out on a Poros R1 1×150 mm column (10 μm) (Applied Biosystems). The flow was fixed at 0.4 mL/min, column temperature was maintained at 50° C. and the gradient used was as follows: solvent A: acidified water (0.1% TFA), solvent B: acidified acetonitrile (0.1% TFA). The gradient begins at 5% of solvent B, is maintained for 5 minutes, is then increased linearly to 85% over 25 minutes and is then increased again to 95% over one minute and maintained at 95% for 3 minutes. A step of equilibration of the column in 5% of solvent B follows elution. Elution is monitored at 210 nm (FIG. 7).

### 7.2 MALDI-TOF MS Analyses

MALDI-TOF analyses were carried out on the antibody. The solution of antibody is desalted on ZipTip C18 (Millipore). A quantity of 70 pmol of antibody is loaded and then eluted in 5 μL H₂O/acetonitrile/HCOOH 20/75/5 (v/v/v). 0.6 μL of the sample (about 8 pmol) is deposited in triplicate on an MTP 348 MALDI plate (Brucker Daltonics) with 0.6 μL of sinapinic acid (at 2 mg/mL in 50% of acetonitrile). The analyses were carried out in positive mode (FIG. 8) and the mass spectrometer (Autoflex, Brucker Daltonics) was calibrated over an m/z range from 20000 to 190000 with BSA.

This result allowed an estimate of the mass of the dreg55 antibody around 148000 Da.

### 7.3 Labelling of the Antibody

The labelling experiment was carried out on 400 μg (100 μL of a solution at 4 mg/mL i.e. 2.7×10⁻⁹ mol) of the dreg55 antibody, to which 0.65 mg of solid ligand L₂ (3.4×10⁻⁸ mol of ligand L₂ containing 17% of lutidinium salts) was added, which means 230 equivalents of ligand L₂ for each antibody. The pH was adjusted to 7.3 with PBS buffer and the reaction mixture was stirred for one hour at ambient temperature.

The reaction mixture was then purified in order to remove the excess ligand L₂ by ultrafiltration on vivaspin500 modules (Sartorius) with a cut-off of 30 kDa and washed several times (20 cycles) with a Tris-HCl 0.01 M buffer at pH 7.4.

The reaction mixture (11 pmol) was then deposited, after desalting on ZipTip C18, on a MALDI plate as described above (FIG. 9).

The mass spectrum shows a shift of the [M+H]⁺ peak to the right due to the mass of the ligand L₂, which is added to that of the antibody. At the level of the maximum, there is a difference of 7000 Da, which would correspond to approximately 9 ligands L₂ per antibody.

### 7.4 Fluorescence Assay

An estimate of the number of ligands L₂ per antibody was also established by a fluorescence assay. The assay was carried out on 6.7×10⁻¹¹ mol of the labelled antibody by successive additions of a solution of TbCl₃ at 5×10⁻⁶ M prepared in a solution of Tris-HCl 0.01 M pH 7 (20 μL/addition).

The measurements were carried out on a Horiba Jobin Yvon spectrofluorometer using a filter at 390 nm and an excitation wavelength λ_{excitation}=328 nm. The emission spectrum was acquired from 400 to 750 nm and the reading was taken at the peak emission of terbium at 544 nm.

A linear increase in fluorescence measured at 544 nm is observed followed by a plateau that begins at around 2×10⁻⁹ mol of TbCl₃ (FIG. 10). The intersection of the two straight lines corresponds to the equivalence point of the assay. The ratio of this quantity to the quantity of antibody present in solution gives the number of ligands L₂ per antibody. This number is evaluated at 30 ligands L₂/antibody.

7.5 Complexing with Terbium

The labelled antibody was then complexed with Tb³⁺ by adding 3 equivalents of a solution of TbCl₃ at 5.35×10⁻⁴ M in a solution of Tris-HCl 0.01 M pH 7. The reaction mixture was purified again by ultrafiltration (vivaspin500, cut-off 30 kDa) and was washed several times (20 cycles) with a Tris-HCl 0.01 M buffer at pH 7 in order to remove the excess Tb³⁺.

7.6 Characterization of the Labelled dreg55 Antibody by Fluorescence

The measurements were carried out on a Horiba Jobin Yvon Fluoromax spectrofluorometer in a solution of Tris-HCl 0.01 M at pH 7.

7.6.1 Emission

The excitation wavelength used for measuring the emission spectrum was 328 nm. Acquisition was carried out from 400 to 750 nm, and maximum emission was measured at 544 nm (FIG. 11).

7.6.2 Excitation

The excitation spectrum was measured relative to an emission at 544 nm and was acquired between 250 and 450 nm (FIG. 12).

The excitation spectrum shows a maximum at 328 nm.

7.6.3 Lifetime

The lifetime of the complex was measured using the excitation and emission wavelengths established previously (λ_{excitation}=328 nm and λₑₘᵢₛₛᵢₒₙ=544 nm) (FIG. 13).

The curve shows a monoexponential decrease in luminescence, which indicates the presence of a single species that emits in solution. The measured lifetime is 1.6 ms.

### EXAMPLE 8

### Labelling of the dreg200 Antibody with the Ligand L₂

The dreg200 antibody is a mouse antibody directed against L-selectin.

### 8.1 MALDI-TOF MS Analyses

A MALDI-TOF analysis was carried out on the antibody. The solution of antibody was desalted on ZipTip C18 and then deposited on a MALDI target as described above. The result is shown in FIG. 14.

The MALDI mass spectrum shows that the dreg200 antibody has a molar mass of about 150000 Da.

8.2 Labelling of dreg200

The labelling experiment was carried out on 200 μg (25 μL of a solution at 8 mg/mL i.e. 1.32×10⁻⁹ mol) of the dreg200 antibody, to which 0.27 mg of solid ligand L₂ (2.58×10⁻⁷ mol of ligand L₂ containing 17% of lutidinium salts) was added, which means 200 equivalents of ligand L₂ for each antibody. The pH was adjusted to 7.3 with PBS buffer and the reaction mixture was stirred for one hour at ambient temperature.

The reaction mixture was then purified in order to remove the excess ligand L₂ by ultrafiltration on vivaspin500 modules with a cut-off of 30 kDa and washed several times (20 cycles) with a Tris-HCl 0.01 M buffer at pH 7.

The reaction mixture (11 pmol) was then deposited after desalting by ZipTip C18 on a MALDI plate as described above. The result is shown in FIG. 15. The MALDI spectrum shows a shift of the [M+H]⁺ peak to the right, of 8000 Da. This mass difference corresponds to about 10 ligands L₂/antibody.

### 8.3 Fluorescence Assay

An estimate of the number of ligands L₂ per antibody was also established by a fluorescence assay. The assay was carried out on 3.8×10⁻¹¹ mol of the labelled antibody by successive additions of a solution of TbCl₃ at 10⁻⁶ M prepared in a solution of Tris-HCl 0.01 M at pH 7 (20 μL/addition).

A linear increase in fluorescence measured at 544 nm is observed followed by a plateau that begins at around 8×10⁻¹⁰ mol of TbCl₃ (FIG. 16). The intersection of the two straight lines corresponds to the equivalence point of the assay. The ratio of this quantity to the quantity of antibody present in solution gives the number of ligands L₂ per antibody. This number is evaluated at 21 ligands L₂/antibody.

8.4 Complexing with Terbium

The labelled antibody was then complexed with Tb³⁺ by adding 2 equivalents of a solution of TbCl₃ at 7.4×10⁻⁵ M in a solution of Tris-HCl 0.01 M at pH 7. The reaction mixture was purified again by ultrafiltration (vivaspin500, cut-off 30 kDa) and was washed several times (20 cycles) with Tris-HCl 0.01 M buffer at pH 7 in order to remove the excess Tb³⁺.

### EXAMPLE 9

### Labelling of L-Selectin with the Ligand L₂

The experiments were carried out on 50 μg of L-selectin (recombinant human protein).

### 9.1 HPLC Analysis

A method was developed for analysing L-selectin by HPLC. The analyses were carried out on an Alliance 2695 HPLC chain (Waters) equipped with a UV detector with a Waters 2996 diode array. The separations were carried out on a Symmetry C18 column 3.5 μm, 4.6×75 mm (Waters). The flow was fixed at 0.5 mL/min, column temperature was maintained at 40° C. and the gradient used was as follows: solvent A: acidified water (0.1% TFA), solvent B: acidified acetonitrile (0.1% TFA). The gradient begins at 5% of solvent B, is maintained for 5 minutes and is then increased linearly to 85% over 25 minutes and is then increased again to 95% over one minute and maintained at 95% for 3 minutes. A step of equilibration of the column in 5% of solvent B follows elution. Elution is monitored at 210 nm (FIG. 17).

### 9.2 MALDI-TOF MS

A MALDI-TOF analysis was carried out on the sample. The solution of antibody was desalted on ZipTip C18 and then deposited on a MALDI target as described above. The [M+H]⁺ peak obtained is relatively broad and shows a mass of 72 kDa for L-selectin (FIG. 18).

### 9.3 Labelling of L-Selectin

The labelling experiment was carried out on a quantity of 46 μg (6.4×10⁻⁹ mol) of L-selectin, to which 10 μL of a solution of ligand L₂ is added at 1.02 mg/mL (containing 17% of lutidinium salts) prepared in 0.005% HCl (pH ˜3), which means 15 equivalents of ligand L₂ for each equivalent of L-selectin. The pH was adjusted to 7.3 with PBS buffer and the reaction mixture was stiffed for one hour at ambient temperature.

The reaction mixture was then purified in order to remove the excess ligand L₂ on Zeba columns (Thermo Scientific), cut-off 7 kDa (a single passage). The columns were prepared with a Tris-HCl 0.01 M buffer at pH 7.

The reaction mixture was then deposited after desalting by ZipTip C18 on a MALDI target as described above (FIG. 19).

The MALDI spectrum shows a shift of the [M+H]⁺ peak to the right, of 2000 Da. This mass difference corresponds to about 3 ligands L₂/L-selectin.

9.4 Complexing with Terbium

The sample of labelled L-selectin was then complexed with Tb³⁺ by adding 5 equivalents of a solution of TbCl₃ at 10⁻³ M in a solution of Tris-HCl 0.01 M at pH 7. The reaction mixture was purified again on a Zeba column (a single passage) prepared with a Tris-HCl 0.01M buffer at pH 7 in order to remove the excess Tb³⁺.

### EXAMPLE 10

### Labelling of the PSS233 Antibody with the Ligand L₂

The PSS233 antibody is an anti-PSA (Prostate Specific Antigen) antibody.

### 10.1 MALDI-TOF MS Analysis

A MALDI-TOF analysis was carried out on the antibody before labelling in order to determine its molar mass. The antibody was desalted on ZipTip C18 and then deposited on a MALDI target as described above using sinapinic acid as matrix (FIG. 20).

The MALDI-MS analysis shows that the PSS233 antibody has a molar mass of 150000 Da.

### 10.2 Labelling of PSS233

The labelling experiment was carried out on 108 μg (7.2×10⁻¹⁰ mol) of PSS233, to which 0.8 mg of solid ligand L₂ (7.7×10⁻⁷ mol of ligand L₂ containing 17% of lutidinium salts) was added, which means 1065 equivalents of ligand L₂ for each antibody. The pH was adjusted to 7.1 with PBS buffer and the reaction mixture was stirred for one hour at ambient temperature.

The reaction mixture was then purified in order to remove the excess ligand L₂ by ultrafiltration on vivaspin500 modules with a cut-off of 30 kDa and washed several times (20 cycles) with a Tris-HCl 0.01 M buffer at pH 7.

The reaction mixture was then deposited after desalting by ZipTip C18 on a MALDI plate as described above (FIG. 21).

The MALDI spectrum shows a shift of the [M+H]⁺ peak of 5500 Da to the right. This mass difference corresponds to about 7 ligands L₂/antibody.

10.3 Complexing with Terbium

The labelled antibody was then complexed with Tb³⁺ by adding 2.2×10⁻⁸ mol of a solution of TbCl₃ in a solution of Tris-HCl 0.01 M pH 7. The reaction mixture was purified again by ultrafiltration (vivaspin500, cut-off 30 kDa) and was washed several times (20 cycles) with Tris-HCl 0.01 buffer pH 7 in order to remove the excess Tb³⁺.

### 10.4 Characterization of the Labelled PSS233 Antibody by Fluorescence

The measurements were carried out on a Horiba Jobin Yvon Fluoromax spectrofluorometer in Tris-HCl 0.01 M buffer at pH 7.

10.4.1 Emission

The excitation wavelength used for measuring the emission spectrum was 328 nm

Acquisition took place from 400 to 750 nm, and maximum emission was measured at 544 nm (FIG. 22).

10.4.2 Excitation

The excitation spectrum was measured relative to an emission at 544 nm and was acquired between 250 and 450 nm (FIG. 23).

The excitation spectrum shows a maximum at 329 nm.

10.4.3 Lifetime

The lifetime of the complex was measured using the excitation and emission wavelengths established previously (λ_{excitation}=328 nm and λₑₘᵢₛₛᵢₒₙ=544 nm) (FIG. 24).

The curve shows a monoexponential decrease in luminescence, which indicates the presence of a single species that emits in solution. The lifetime measured is 1.94 ms.

### EXAMPLE 11

### Labelling of the PSR222 Antibody with the Ligand L₂

The PSR222 antibody is an anti-PSA (Prostate Specific Antigen) antibody.

### 11.1 MALDI-TOF MS Analysis

A MALDI-TOF analysis was carried out on the antibody before labelling in order to determine its molar mass. The antibody was desalted on ZipTip C18 and then deposited on a MALDI target as described above using sinapinic acid as matrix (FIG. 25).

The MALDI-MS analysis shows that the PSR222 antibody has a molar mass of 147500 Da.

### 11.2 Labelling of PSR222

The labelling experiment was carried out on 75 μg (5×10⁻¹⁰ mol) of PSR222, to which 0.13 mg of solid ligand L₂ (1.25×10⁻⁷ mol of ligand L₂ containing 17% of lutidinium salts) was added, which means 250 equivalents of ligand L₂ for each antibody. The pH was adjusted to 7.1 with PBS buffer and the reaction mixture was stiffed for one hour at ambient temperature.

The reaction mixture was then purified in order to remove the excess ligand L₂ by ultrafiltration on vivaspin500 modules with a cut-off of 30 kDa and washed several times (20 cycles) with a Tris-HCl 0.01 M buffer at pH 7.

The reaction mixture was then deposited, after desalting on ZipTip C18, on a MALDI plate as described above (FIG. 26).

The MALDI spectrum shows a shift of the [M+H]⁺ peak of 2500 Da to the right. This mass difference corresponds to about 3 ligands L₂/antibody.

11.3 Complexing with Terbium

The labelled antibody was then complexed with Tb³⁺ by adding 5.25×10⁻⁹ mol of a solution of TbCl₃ in a solution of Tris-HCl 0.01 M at pH 7. The reaction mixture was purified again by ultrafiltration (vivaspin500, cut-off 30 kDa) and was washed several times (20 cycles) with Tris-HCl 0.01 M buffer at pH 7 in order to remove the excess Tb³⁺.

### 11.4 Characterization of the Labelled PSR222 Antibody by Fluorescence

The measurements were carried out on a Horiba Jobin Yvon Fluoromax spectrofluorometer in Tris-HCl 0.01 M buffer at pH 7.

11.4.1 Emission

The excitation wavelength used for measuring the emission spectrum was 328 nm. Acquisition took place from 400 to 750 nm, and maximum emission was measured at 544 nm (FIG. 27).

11.4.2 Excitation

The excitation spectrum was measured relative to an emission at 544 nm and was acquired between 250 and 450 nm (FIG. 28).

The excitation spectrum shows a maximum at 329 nm

11.4.3 Lifetime The lifetime of the complex was measured using the excitation and emission wavelengths established previously (λ_{excitation}=328 nm and λₑₘᵢₛₛᵢₒₙ=544 nm) (FIG. 29).

The curve shows a monoexponential decrease in luminescence, which indicates the presence of a single species that emits in solution. The lifetime measured is 2.17 ms.

### EXAMPLE 12

### Labelling of the EgB4 Antibody Fragment with the Ligand L₂

EgB4 is an antibody fragment (consisting of a single variable monomeric domain of an antibody) directed against the epidermal growth factor (EGF).

### 12.1 MALDI-TOF MS Analysis

A MALDI-TOF analysis was carried out on the antibody fragment. The solution of antibody fragment was desalted on ZipTip C18 and then a quantity of 15 pmol of the sample was deposited on a MALDI target using sinapinic acid as matrix. Acquisition was carried out in positive mode over the m/z range from 4000 to 40000. The mass spectrometer was calibrated using myoglobin (FIG. 30).

The MALDI analysis shows that the EgB4 antibody fragment has a molar mass of 17200 Da.

### 12.2 Labelling of the EgB4 Antibody Fragment

Labelling was carried out on 270 μg (1.57×10⁻⁸ mol) of the EgB4 antibody fragment, to which 1.58 mg of solid ligand L₂ (1.33×10⁻⁶ mol of ligand L₂ containing 27% of lutidinium salts) was added, which means 85 equivalents of ligand L₂ for each antibody fragment. The pH was adjusted to 7 with PBS buffer and the reaction mixture was stiffed for one hour at ambient temperature.

The reaction mixture was then purified in order to remove the excess ligand L₂ by ultrafiltration on vivaspin500 modules with a cut-off of 10 kDa and washed several times (20 cycles) with a Tris-HCl 0.01 M buffer at pH 7.

The reaction mixture was then deposited after desalting by ZipTip C18 on a MALDI plate as described above, but no signal could be obtained.

### 12.3 Tricine Gel SDS-PAGE

In order to gain an idea of the degree of labelling, the labelled antibody fragment was deposited on a tricine SDS polyacrylamide gel. The gels were prepared according to a procedure described by H. Schagger and G. Von Jagow (Analytical Biochemistry, 1987, 166, 368). Replacing the glycine in the conventional gels with tricine allows better separation of the proteins of low molecular weight. The spacer gel described by H. Schagger and G. Von Jagow was omitted and the acrylamide concentration used for the gels was 16.5% T.

Before deposition on the gel, the sample is diluted (with a ratio of at least 1:4) in a Laemmli buffer, preparation of which is described by U. K. Laemmli (Nature, 1970, 277, 680).

The gel shows that the antibody fragment was completely labelled and that the labelled product has a mass distribution between 19 and 25 kDa, which would correspond to a degree of labelling between 2 and 10 ligands L₂/antibody fragment.

12.4 Complexing with Terbium

The solution of labelled antibody fragment was then complexed with Tb³⁺ by adding 2.35×10⁻⁷ mol of a solution of TbCl₃ in 0.01 M Tris-HCl pH 7, or 15 equivalents of Tb³⁺. The reaction mixture was purified again by ultrafiltration (vivaspin500, cut-off 10 kDa) and was washed several times (20 cycles) with Tris-HCl 0.01 M at pH 7 to remove the excess Tb³⁺. A precipitate formed after adding the solution of Tb³⁺.

### EXAMPLE 13

### Labelling of the EgA1 Antibody Fragment with the Ligand L₂

EgA1 is an antibody fragment directed against the epidermal growth factor (EGF).

### 13.1 MALDI-TOF MS Analysis

A MALDI-TOF analysis was carried out on the antibody fragment. The solution of antibody fragment was desalted on ZipTip C18 and then a quantity of 15 pmol of the sample was deposited on a MALDI target using sinapinic acid as matrix (FIG. 32).

MALDI analysis shows that the EgA1 antibody fragment has a molar mass of 17100 Da.

### 13.2 Labelling of the EgA1 Antibody Fragment

Labelling was carried out on 115 μg (6.7×10⁻⁹ mol) of the EgA1 antibody fragment, to which 1.38 mg of solid ligand L₂ (1.1×10⁻⁶ mol of ligand L₂ containing 27% of lutidinium salts) was added, which means 170 equivalents of ligand L₂ for each antibody fragment. The pH was adjusted to 7 with PBS buffer and the reaction mixture was stiffed for one hour at ambient temperature.

The reaction mixture was then purified in order to remove the excess ligand L₂ by ultrafiltration on vivaspin500 modules with a cut-off of 10 kDa and washed several times (20 cycles) with a Tris-HCl 0.01 M buffer at pH 7.

The reaction mixture was then deposited, after desalting on ZipTip C18, on a MALDI target as described above, but no signal could be obtained.

### 13.3 SDS-PAGE

13.3.1 Tricine Gel SDS-PAGE

In order to gain an idea of the degree of labelling, the labelled antibody fragment was deposited on a tricine SDS polyacrylamide gel. The gels were prepared as described above and the acrylamide concentration used for the gels was 16.5% T.

Before deposition on the gel, the sample is diluted (with a ratio of at least 1:4) in a Laemmli buffer.

As shown in FIG. 33, the gel shows no variation of mass at the level of the labelled antibody fragment. A fraction of the antibody fragment seems to have a lower mass than the unlabelled antibody fragment. No conclusion could be drawn from this gel.

13.3.2 Glycine SDS-PAGE Gel

A glycine SDS-PAGE gel was also prepared for this sample with an acrylamide concentration of 15% T. Before deposition on the gel, the sample is diluted (with a ratio of at least 1:4) in a Laemmli buffer.

The gel (FIG. 34) shows that the antibody fragment was completely labelled and that the labelled product has a mass distribution between 18 and 24 kDa, which would correspond to a degree of labelling between 1 and 10 ligands L₂/antibody fragment.

13.4 Complexing with Terbium

The solution of labelled antibody fragment was then complexed with Tb³⁺ by adding 1×10⁻⁷ mol of a solution of TbCl₃ in a solution of Tris-HCl 0.01 M at pH 7, or 15 equivalents of Tb³⁺. The reaction mixture was purified again by ultrafiltration (vivaspin500, cut-off 10 kDa) and was washed several times (20 cycles) with Tris-HCl 0.01 buffer at pH 7 in order to remove the excess Tb³⁺. A precipitate formed after adding the solution of Tb³⁺.

1. Compounds of the following Formula (I): 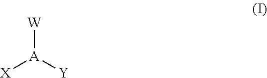
in which
A represents
either a nitrogen atom,
or a ring comprising from 3 to 6 carbon atoms, or an aromatic ring comprising from 5 to 10 members, said ring and said aromatic ring optionally comprising one or more heteroatoms selected from N, O and S,
W represents
either a bromine atom, or an iodine atom,
or an E-G-Q group with
E selected from the group consisting of an oxygen atom, a —C≡C— group, a (CH₂)ₘ group, m being an integer comprised between 0 and 5, and a —CONH— group,
G is selected from the group consisting of
i) —(CH₂)ₒ, o being an integer comprised between 0 and 5,
ii) —(CH₂)ₙ—NH—, n being an integer comprised between 0 and 5,
iii) —(CH₂)p-CO—NH—(CH₂)q-NH—, p and q being integers comprised between 0 and 5, and
iv) 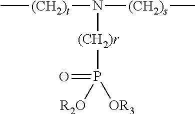
r, s and t being, each independently of one another, an integer comprised between 0 and 5, and R₂ and R₃ representing, each independently of one another, a hydrogen atom, a (C₁-C₄)alkyl group or a hydrolyzable group
Q represents either a hydrogen atom, or an amine protecting group, or a functional group capable of forming a covalent bond with the primary and secondary amines, alcohols and thiols and
X and Y represent, independently of one another,
either a hydrogen atom,
or a 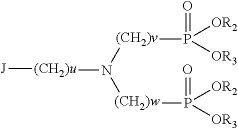
group, where u is an integer equal to 0 or 1, v and w, identical or different, are integers equal to 1 or 2, R₂ and R₃ represent, each independently of one another, a hydrogen atom, a (C₁-C₄)alkyl group or a hydrolyzable group selected from the group consisting of the esters and the amides and J is either —CH₂, or is an aromatic ring comprising from 5 to 10 members and optionally one or more heteroatoms selected from N, O and S,
or a 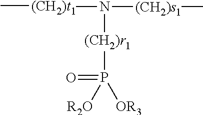
group, where r₁, s₁ and t₁ are, each independently of one another, an integer comprised between 1 and 2, and R₂ and R₃ represent, each independently of one another, a hydrogen atom, a (C₁-C₄)alkyl group or a hydrolyzable group selected from the group consisting of the esters and the amides,
provided that X, W and Y are not simultaneously H,
and salts thereof.

2. The compounds according to claim 1, wherein:
X and Y, or
X, W and Y,
are not simultaneously H,
and the compounds of the following formula: 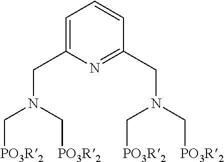
in which R′ represents H or Et, are excluded.

3. The compounds according to claim 2, wherein A represents an aromatic ring comprising from 5 to 10 members and optionally one or more heteroatoms selected from N, O and S.

4. The compounds according to claim 3, wherein A represents a pyridine.

5. The compounds according to claim 4, characterized in that X and Y are in a position ortho to the nitrogen of the pyridine and W is in a position para to the nitrogen of the pyridine.

6. The compounds according to claim 1, characterized in that W is selected from the group consisting of:
Br,
—O—(CH₂)₃NHBoc,

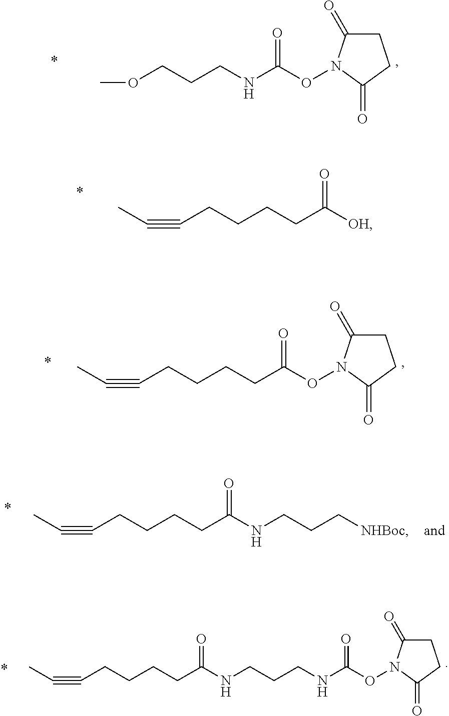

7. The compounds according to claim 1, characterized in that X and Y each represent a 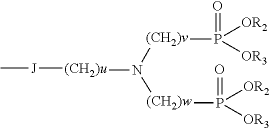
group.

8. The compounds according to claim 1, wherein J represents a pyrazol-1-yl group or a pyridin-2-yl group.

9. The compounds according to claim 4, characterized in that X and W are in a position ortho to the nitrogen of the pyridine and Y is in a position para to the nitrogen of the pyridine.

10. The compounds according to claim 9, wherein W represents an E-G-Q group, wherein:
E representing a (CH₂)ₘ group, m being an integer comprised between 0 and 5
G representing, in the group comprising
i) 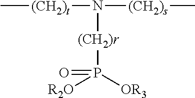
r, s and t being, each independently of one another, an integer comprised between 0 and 5, and R₂ and R₃ representing, each independently of one another, a hydrogen atom, a (C₁-C₄)alkyl group or a hydrolyzable group,
Q representing either a hydrogen atom, or an amine protecting group, or a functional group capable of forming a covalent bond with the primary and secondary amines, alcohols and thiols,
X represents: 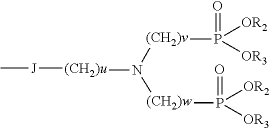
and Y represents H.

11. A method for preparing a compound of Formula (I) according to claim 1, comprising a step of selective deprotection of the phosphonic esters, said step comprising bringing a compound of Formula (I) bearing an activated function and at least one phosphonic ester function into contact with trimethylsilyl bromide in a solvent such as dichloromethane or chloroform in the presence of lutidine followed by a deprotection of the silylated esters with an alcohol, in particular methanol.

12. A complex between a metal ion and a bifunctional ligand according to claim 1.

13. A complex according to claim 12, characterized in that it is in addition conjugated with a target structure selected from the group comprising biologically active compounds or a carrier and in that Formula (I) 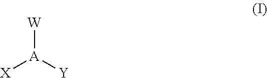
is that in which
A represents
either a nitrogen atom,
or a ring comprising from 3 to 6 carbon atoms, or an aromatic ring comprising from 5 to 10 members, said ring and said aromatic ring optionally comprising one or more heteroatoms selected from N, O and S,
W represents
an E-G-Q group with
E selected from the group comprising an oxygen atom, a —C≡C— group, a (CH₂)ₘ group, m being an integer comprised between 0 and 5 and a —CONH— group,
G is selected from the group consisting of
i) —(CH₂)ₒ, o being an integer comprised between 0 and 5,
ii) —(CH₂)ₙ—NH—, n being an integer comprised between 0 and 5,
iii) —(CH₂)p-CO—NH—(CH₂)q-NH—, p and q being integers comprised between 0 and 5,
iv) —(CH₂)ₘ—CO—NH—(CH₂)ₚ—NHZ and
v) 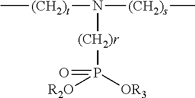
r, s and t being, each independently of one another, an integer comprised between 0 and 5, and R₂ and R₃ represent, each independently of one another, a hydrogen atom, a (C₁-C₄)alkyl group or a hydrolyzable group
Q a functional group capable of forming a covalent bond with the primary and secondary amines, alcohols and thiols, and
X and Y represent, independently of one another,
either a hydrogen atom,
or a 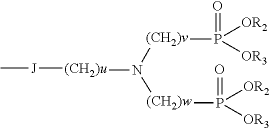
group, where u is an integer equal to 0 or 1, v and w, identical or different, are integers equal to 1 or 2, R₂ and R₃ represent, each independently of one another, a hydrogen atom, a (C₁-C₄)alkyl group or a hydrolyzable group selected from the group consisting of the esters and the amides and J is either —CH₂, or is an aromatic ring comprising from 5 to 10 members and optionally one or more heteroatoms selected from N, O and S,
or a 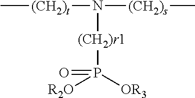
group, where r₁, s₁ and t₁ are, each independently of one another, an integer comprised between 1 and 2, and R₂ and R₃ represent, each independently of one another, a hydrogen atom, a (C₁-C₄)alkyl group or a hydrolyzable group selected from the group comprising the esters and the amides provided that X, W and Y are not simultaneously H,
and pharmaceutically acceptable salts thereof, thus constituting a conjugated complex.

14. A conjugated system comprising a bifunctional ligand of Formula (I) as defined in claim 10 and a biologically active compound or a carrier.

15. A complex according to claim 12, wherein the bifunctional ligand is selected from the group comprising L1, 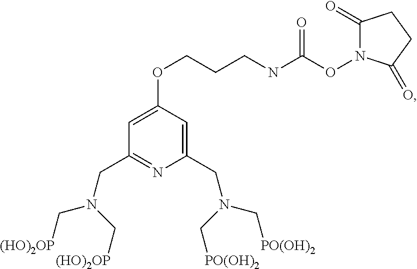
L2: 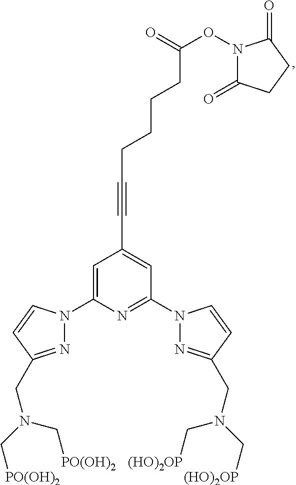
L3: 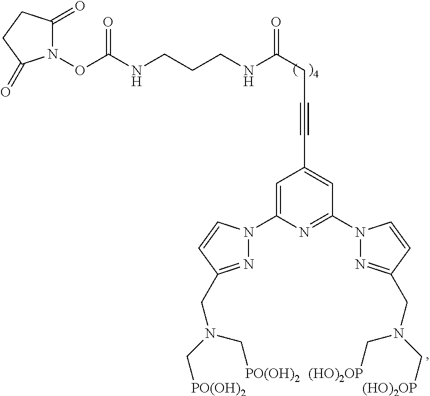
and L4:

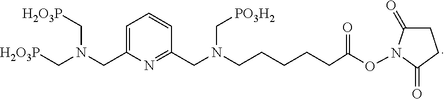

16. A diagnostic agent comprising at least one complex according to claim 12 and a pharmaceutically acceptable vehicle.

17. (canceled)

18. A kit for preparing a diagnostic agent comprising a bifunctional ligand as defined in claim 10.

19. The kit for preparing a diagnostic agent according to claim 18, comprising:
(1) said bifunctional ligand; and
(2) a solution of a salt or of a chelate of a metallic radionuclide.

20. A conjugated system according to claim 14, wherein the bifunctional ligand is selected from the group comprising L1, 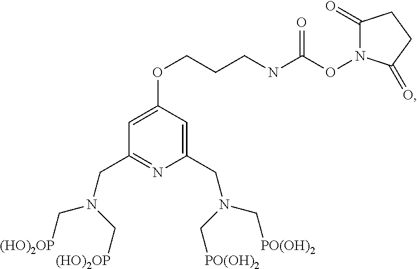
L2: 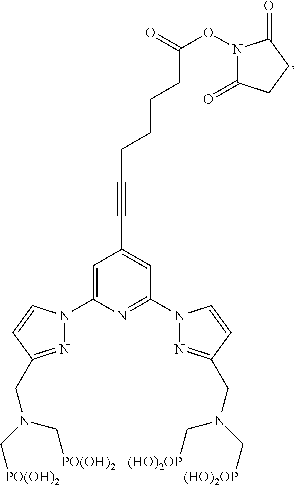
L3: 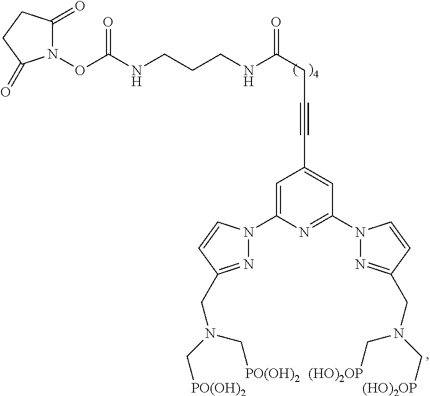
and L4:

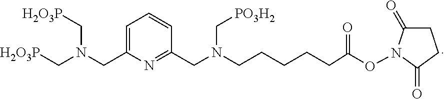

21. A diagnostic agent comprising at least one system according to claim 14 and a pharmaceutically acceptable vehicle.
